(19) United States
(12) Patent Application Publication
(10) Pub. No.: US 2020/0396411 A1
(43) Pub. Date: Dec. 17, 2020

(54) INFORMATION PROCESSOR, INFORMATION PROCESSING METHOD, AND PROGRAM

(71) Applicant: SONY CORPORATION, TOKYO (JP)

(72) Inventors: HIRONORI HATTORI, TOKYO (JP); SHO OGURA, KANAGAWA (JP)

(21) Appl. No.: 16/761,469

(22) PCT Filed: Aug. 30, 2018
(86) PCT No.: PCT/JP2018/032189

§ 371 Date: May 4, 2020

(30) Foreign Application Priority Data
Nov. 13, 2017 (JP) 2017-218444

(51) Int. Cl. H04N 5/445; G06K 9/00; G06T 7/70; G06T 7/50; H04N 5/262; H04N 5/272; A63B 71/06; A61B 5/107; A61B 5/00

(52) U.S. Cl. CPC H04N 5/445; A63B 69/002; G06T 7/70; G06T 7/50; H04N 5/2628; H04N 5/272; G06K 9/00624; A63B 71/0622; A61B 5/107; A61B 5/4561; G06T 2207/30196; G06T 2207/20092; G06K 2209/27; G06T 2207/30221; A63B 2071/0647; G06K 9/00362

(57) ABSTRACT

[Object] An information processor, an information processing method, and a program.

[Solution] An information processor comprising an output control section that causes an output image obtained in a manner corresponding to each of a plurality of input images to be outputted to reduce a difference in at least one of subject parameters for a subject included in each of the plurality of input images.

### TECHNICAL FIELD

The present disclosure relates to an information processor, an information processing method, and a program.

### BACKGROUND ART

For enhancement in sports, it is important to objectively analyze his or her own play and to play with consciousness for improvement. For this purpose, for example, it has been widely common to record a play as an image (still image or moving image) and to view the image in which the play is recorded (hereinafter, also referred to as a playing image) after the play, thus allowing improvement points and the like to be grasped.

In addition, in order to compare a form of a professional sports player and his or her own form with each other, it has also been common to make comparison between a playing image of the professional sports player and his or her own playing image. For such a purpose, PTL 1 listed below describes a technique of causing a plurality of images to temporally coincide with one another at a designated reproduction point for simultaneous reproduction and displaying.

### CITATION LIST

### Patent Literature

PTL 1: Japanese Unexamined Patent Application Publication No. H10-304299

### SUMMARY OF THE INVENTION

### Problem to be Solved by the Invention

However, in a case where appearances of subjects included (reflected) in respective images are different, there is a possibility that it may be difficult to make comparison among the plurality of images. Therefore, the present disclosure proposes a novel and improved information processor, information processing method, and program that make it possible to make comparison more easily among the plurality of images.

### Means for Solving the Problem

According to the present disclosure, there is provided an information processor comprising an output control section that causes an output image obtained in a manner corresponding to each of a plurality of input images to be outputted to reduce a difference in at least one of subject parameters for a subject included in each of the plurality of input images.

In addition, according to the present disclosure, there is provided an information processing method comprising causing an output image obtained in a manner corresponding to each of a plurality of input images to be outputted to reduce a difference in at least one of subject parameters for a subject included in each of the plurality of input images.

In addition, according to the present disclosure, there is provided a program that causes a computer to implement a function of causing an output image obtained in a manner corresponding to each of a plurality of input images to be outputted to reduce a difference in at least one of subject parameters for a subject included in each of the plurality of input images.

### Effect of the Invention

As described above, according to the present disclosure, it is possible to make comparison more easily among a plurality of images.

It is to be noted that the above-mentioned effects are not necessarily limitative. In addition to or in place of the above effects, there may be achieved any of the effects described in the present specification or other effects that may be grasped from the present specification.

### BRIEF DESCRIPTION OF THE DRAWINGS

FIG. 1 is a block diagram illustrating a schematic configuration of an information processing system according to a first embodiment of the present disclosure.

FIG. 2 is a schematic diagram schematically illustrating an example in which an information processing system 1000 is applied in a practice of a soccer team.

FIG. 3 is a block diagram illustrating a configuration example of an information processor 1 according to the same embodiment.

FIG. 4 is a block diagram illustrating a configuration example of an operation terminal 2 according to the same embodiment.

FIG. 5 is a flowchart diagram illustrating an operation example of the same embodiment.

FIG. 6 is an explanatory diagram that describes Modification Example 1 according to the same embodiment.

FIG. 7 is an explanatory diagram that describes Modification Example 2 according to the same embodiment.

FIG. 8 is an explanatory diagram that describes Modification Example 3 according to the same embodiment.

FIG. 9 is an explanatory diagram that describes Modification Example 3 according to the same embodiment.

FIG. 10 is an explanatory diagram that describes Modification Example 3 according to the same embodiment.

FIG. 11 is an explanatory diagram that describes Modification Example 4 according to the same embodiment.

FIG. 12 is an explanatory diagram that describes Modification Example 4 according to the same embodiment.

FIG. 13 is an explanatory diagram that describes Modification Example 5 according to the same embodiment.

FIG. 14 is an explanatory diagram that describes Modification Example 5 according to the same embodiment.

FIG. 15 is an explanatory diagram that describes Modification Example 6 according to the same embodiment.

FIG. 16 is an image diagram that describes an overview of a second embodiment of the present disclosure.

FIG. 17 is a block diagram illustrating a configuration example of an information processor 7 according to the same embodiment.

FIG. 18 is a flowchart diagram illustrating an operation example of the embodiment.

FIG. 19 is a view schematically depicting a general configuration of a surgery room system.

FIG. 20 is a view depicting an example of display of an operation screen image of a centralized operation panel.

FIG. 21 is a view illustrating an example of a state of surgery to which the surgery room system is applied.

FIG. 22 is a block diagram depicting an example of a functional configuration of a camera head and a camera control unit (CCU) depicted in FIG. 21.

FIG. 23 is an explanatory diagram illustrating a hardware configuration example.

### MODES FOR CARRYING OUT THE INVENTION

Hereinafter, description is given in detail of preferred embodiments of the present disclosure with reference to the attached drawings. It is to be noted that, in the present specification and drawings, repeated description is omitted for components substantially having the same functional configuration by assigning the same reference numerals.

In addition, there is also a case where, in the present specification and drawings, a plurality of components having substantially the same functional configurations may be distinguished by assigning different alphabets that follow the same reference numerals. However, in a case where it is unnecessary to particularly distinguish among a plurality of components having substantially the same functional configurations, only the same reference numerals are assigned.

It is to be noted that description is given of in the following order.

### <<1. First Embodiment>>

### <1-1. Overview>

### <1-2. Configuration Example>

### <1-3. Operation Example>

### <1-4. Modification Examples>

### <1-5. Effects>

### <<2. Second Embodiment>>

### <2-1. Overview Example>

### <2-2. Configuration Example>

### <2-3. Operation Example>

### <2-4. Modification Example>

### <2-5. Effects and Supplementals>

### <<3. Application Example>>

### <<4. Hardware Configuration Example>>

### <<5. Closing>>

### 1. First Embodiment

### 1-1. Overview

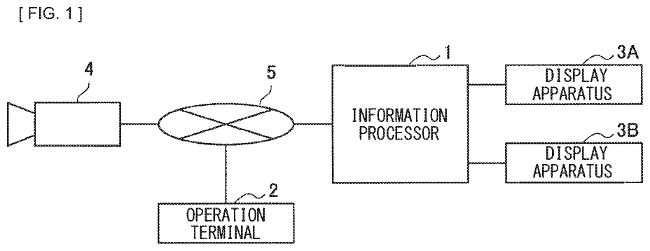

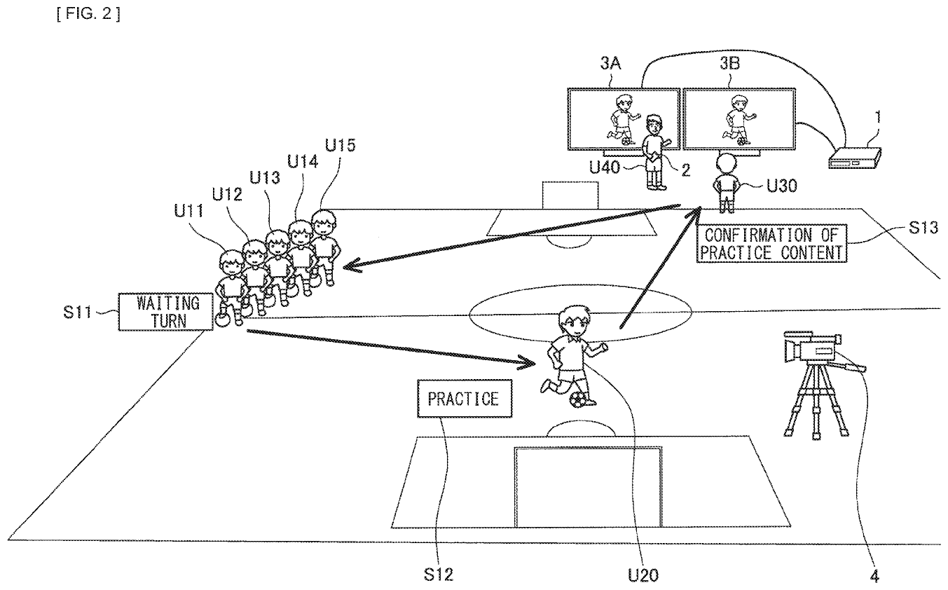

First, description is given of an overview of a first embodiment of the present disclosure with reference to FIG. 1 and FIG. 2. FIG. 1 is a block diagram illustrating a schematic configuration of an information processing system 1000 according to a first embodiment of the present disclosure. As illustrated in FIG. 1, the information processing system 1000 according to the present embodiment includes an information processor 1, an operation terminal 2, a display apparatus 3A, a display apparatus 3B, an imaging apparatus 4, and a communication network 5.

The information processor 1 processes and outputs a plurality of input images for ease of comparison. It is to be noted, the following description of the present embodiment mainly describes an example in which one input image is a moving image having a plurality of frames; however, the input image may be a still image.

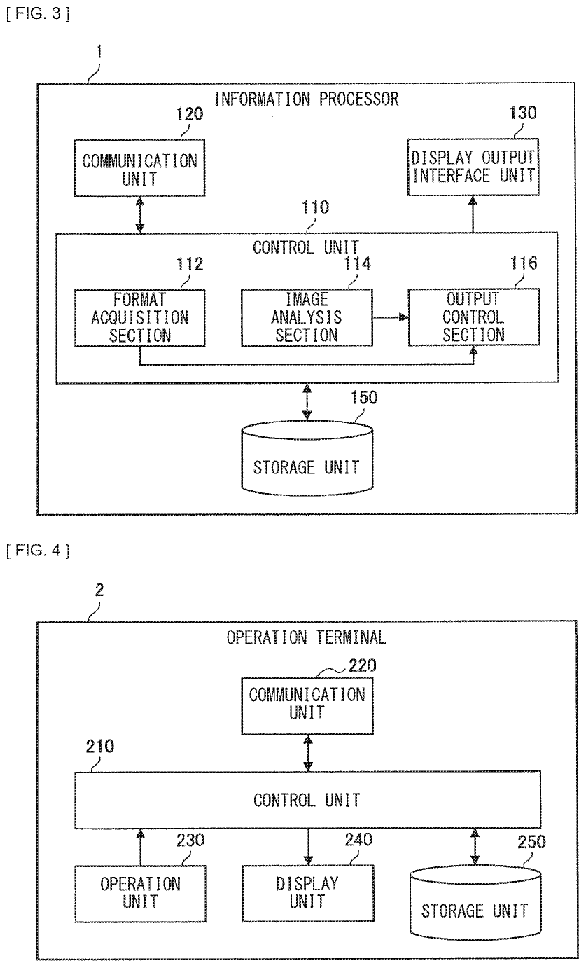

For example, the information processor 1 may perform processing of reducing a difference in appearances of respective subjects included in a plurality of input images, processing of reducing a difference in formats among the plurality of input images, processing of performing adjustment to allow motion timings of the subjects to coincide with one another among moving images, or the like. Respective two output images corresponding to two input images may be separately outputted to the display apparatus 3A and the display apparatus 3B simultaneously. In addition, the plurality of input images may include an image acquired by imaging of the imaging apparatus 4 and received from the imaging apparatus 4 via the communication network 5, or may include an image stored in advance by the information processor 1. It is to be noted that the detailed configuration of the information processor 1 is described later with reference to FIG. 3.

The operation terminal 2 is an information processor that is coupled to the information processor 1 via the communication network 5 to perform operations related to processing performed by the information processor 1. The operation terminal 2 may be, for example, but not limited to, a tablet terminal. A user may operate the operation terminal 2 to thereby select input images to be compared, set a reproduction condition for comparison, for example, specify a key frame related to a motion timing of a subject, or the like. It is to be noted that a detailed configuration of the operation terminal 2 is described later with reference to FIG. 3.

The display apparatus 3A and the display apparatus 3B are each coupled to the information processor 1 by, for example, HDMI (High-Definition Multimedia Interface (registered trademark) or the like, and each display an output image outputted by the information processor 1. The display apparatus 3A and the display apparatus 3B may be arranged side by side. It is to be noted that, in the following, the display apparatus 3A and the display apparatus 3B are each simply referred to as a display apparatus 3 in some cases when there is no need to distinguish them from each other. In addition, FIG. 1 illustrates an example in which two display apparatuses 3 are coupled to the information processor 1, but the number of the display apparatuses 3 coupled to the information processor 1 is not limited to such an example. In addition, FIG. 1 illustrates an example in which the information processor 1 and the display apparatus 3 are directly coupled to each other. However, the information processor 1 and the display apparatus 3 may be coupled to each other via the communication network 5.

The imaging apparatus 4 acquires an image by imaging. In addition, as illustrated in FIG. 1, the imaging apparatus 4 is coupled to the information processor 1 via the communication network 5, and transmits the image acquired by imaging to the information processor 1.

The communication network 5 is a wired or wireless transmission path for information transmitted from an apparatus coupled to the communication network 5. For example, the communication network 5 may include a public network such as the Internet, a telephone network, a satellite communication network, and various types of LAN (Local Area Network) including Ethernet (registered trademark), WAN (Wide Area Network), and the like. In addition, the communication network 5 may include a private network such as IP-VPN (Internet Protocol-Virtual Private Network).

The description has been given above of the schematic configuration of the information processing system 1000 according to the present embodiment. As described above, the information processing system 1000 according to the present embodiment causes the display apparatus 3 to display an output image processed by the information processor 1, in accordance with the operation of the user who uses the operation terminal 2, on a plurality of input images including images acquired by the imaging of the imaging apparatus 4, thereby making it possible to make comparison more easily among a plurality of images.

Next, description is given of an application example of the information processing system 1000 according to the present embodiment described above. FIG. 2 is a schematic diagram schematically illustrating an example in which the information processing system 1000 is applied in a practice of a soccer team.

As illustrated in FIG. 2, users who participate in the practice first wait for their turns for the practice (S11). In the example illustrated in FIG. 2, the users U11 to U15 wait for their turns.

Next, a user whose turn has come (user U20 in the example of FIG. 2) practices a predetermined play (e.g., a shot, etc.) (S12). In step S12, the imaging apparatus 4 captures an image of a practice scene of the user to acquire a playing image, and transmits the image to the information processor 1.

After the practice, a user (user U30 in the example of FIG. 2) receives guidance from another user (user U40 in the example of FIG. 2) such as a coach, for example, while confirming practice contents using the display apparatus 3A and the display apparatus 3B (S13). In this step S13, the information processor 1 may process a playing image of the user U30 and another playing image (e.g., a playing image of a professional player) to be easily compared with each other, and may output the processed playing images to the display apparatus 3A and the display apparatus 3B. Such a configuration enables the user U30 to confirm the practice contents while comparing his own playing image and other playing images with each other more easily. In addition, in step S13, the user U40 may select playing images to be displayed on the display apparatus 3, set a reproduction condition for comparison, or the like through an operation using the operation terminal 2.

Thereafter, the user returns to the waiting for the turn in step S11.

The description has been given above of the application example of the information processing system 1000 according to the present embodiment. It is to be noted that the example has been described above in which the information processing system 1000 according to the present embodiment is applied to the practice of a soccer team; however, the information processing system 1000 according to the present embodiment is not limited to such an example, but can be applied not only to sports other than soccer but also to various scenes other than sports.

### 1-2. Configuration Example

Next, description is given in more detail of the configuration of the information processor 1 and the configuration of the operation terminal 2, among the configurations illustrated in FIG. 1, with reference to FIG. 3 and FIG. 4, respectively.

### (Information Processor)

FIG. 3 is a block diagram illustrating a configuration example of the information processor 1 according to the present embodiment. As illustrated in FIG. 3, the information processor 1 includes a control unit 110, a communication unit 120, a display output interface unit 130, and a storage unit 150.

The control unit 110 functions as an arithmetic processor and a controller, and controls overall operations in the information processor 1 in accordance with various programs. In addition, as illustrated in FIG. 2, the control unit 110 according to the present embodiment has functions as a format acquisition section 112, an image analysis section 114, and an output control section 116.

The format acquisition section 112 acquires a format parameter for an image format of an input image. It is to be noted that, in the present embodiment, the input image may be an image received from another apparatus (e.g., the imaging apparatus 4 illustrated in FIG. 1), or may be an image stored in the storage unit 150 described later.

The format parameter acquired by the format acquisition section 112 may include, for example, a frame rate, resolution, an aspect ratio, or the like. The format acquisition section 112 may acquire the format parameter on the basis of an input image, may acquire the format parameter from another apparatus that provides an input image, or may acquire the format parameter from the storage unit 150.

The format acquisition section 112 may provide the acquired format parameter to the output control section 116 or may cause the storage unit 150 to store the format parameter.

The image analysis section 114 performs analysis of an input image. For example, the image analysis section 114 according to the present embodiment may acquire a subject parameter for a subject included in the input image by analysis of the input image.

The subject parameter as used herein means a parameter that is acquirable, for a subject included in an input image, from the input image. For example, the subject parameter may include a parameter indicating information regarding the subject itself, a parameter indicating relative information on the subject in the input image, a parameter indicating information on a relationship between an imaging apparatus involved with imaging of the input image and the subject, and the like. The subject parameter may include, as parameters indicating information regarding the subject itself, parameters such as a dominant hand of the subject, a dominant foot of the subject, and a size (e.g., height) of the subject, for example. In addition, the subject parameter may include, as the parameter indicating the relative information on the subject in the input image, a parameter such as a position of the subject in the input image. In addition, the subject parameter may include, as the parameter indicating the information on the relationship between the imaging apparatus involved with the imaging of the input image and the subject, parameters such as a distance from the imaging apparatus involved with the imaging of the input image to the subject, and a posture of the subject with respect to the imaging apparatus involved with the imaging of the input image.

It is to be noted that the subject as used herein refers to all of those included in an image; for example, the subject may be a person or a tool such as a ball and a tennis racket, in a case where the present embodiment is applied in the field of sports. The image analysis section 114 may recognize a subject included in an input image by performing analysis of the input image using, for example, a well-known image analysis technique, and may acquire a subject parameter on the basis of information on the recognized subject.

For example, in a case where the image analysis section 114 recognizes that a person and a tennis racket are included in the input image as subjects by analysis of the input image, the parameter of the dominant hand of the subject may be acquired on the assumption that the hand of the person on side holding the tennis racket is the dominant hand. Likewise, in a case where the image analysis section 114 determines that a person and a soccer ball are included in the input image as subjects by analysis of the input image, the parameter of the dominant foot of the subject may be acquired on the assumption that the foot of the person on side kicking the soccer ball is the dominant foot.

The image analysis section 114 may provide the subject parameter acquired in a manner corresponding to each input image to the output control section 116, or may cause the storage unit 150 to store the subject parameter.

In addition, the image analysis section 114 according to the present embodiment may specify a key frame by analysis of an input image including a plurality of frames. The key frame may be, for example, a frame corresponding to a predetermined motion by a subject included in the input image, or may be a frame at a moment when the subject is performing the predetermined motion. For example, in a case where a person who is the subject performs swings of a tennis, the image analysis section 114 may analyze the motion of the subject included in the input image, detect motions such as “take-back”, “impact”, or “follow-through”, and specify a frame corresponding to each motion as a key frame.

It is to be noted that the method of specifying the key frame by image analysis is not particularly limited, but the key frame may be specified on the basis of, for example, a form (posture) of a subject recognized from an input image or an image feature amount extracted from the input image.

The image analysis section 114 may provide the key frame specified in each of input images to the output control section 116, or may cause the storage unit 150 to store the key frame. It is to be noted that, in association with types of the key frame, the key frame may be provided to the output control section 116 or may be stored in the storage unit 150. The types of the key frame may be, for example, types of the predetermined motion in the above-described example, and may be, for example, “take-back”, “impact”, “follow-through”, or the like.

It is to be noted that, although the description has been given above of an example in which the key frame is specified by image analysis performed by the image analysis section 114, the present technology is not limited to such an example. For example, in addition to or in place of the image analysis, the key frame may be specified on the basis of sensor information. For example, the key frame may be specified on the basis of a timing of a whistle detected from audio information (an example of sensor information) acquired by a microphone or motion information (an example of sensor information) acquired by a motion sensor. Alternatively, the key frame may be specified on the basis of an operation of the user acquired via the operation terminal 2; such an example is described later as Modification Example 3.

The output control section 116 outputs an output image on the basis of a plurality of input images to be compared. The output control section 116 may acquire an output image corresponding to each of the plurality of input images. It is to be noted that, here, the plurality of input images to be compared may be selected and determined from among the plurality of input images stored in the storage unit 150 through operations using the operation terminal 2 illustrated in FIG. 1, for example. Alternatively, a plurality of input images to be compared may be automatically selected in accordance with a predetermined condition; for example, one input image stored in advance in the storage unit 150 and an input image newly received from the imaging apparatus 4 may be selected as a plurality of input images to be compared. In addition, as used herein, the phrase “acquire an output image” may include acquiring an input image itself as an output image and acquiring an output image by performing predetermined processing on an input image and by generating the output image.

In addition, the output control section 116 may cause an output image acquired in a manner corresponding to each of the plurality of input images to be outputted to separate apparatuses simultaneously. For example, the output control section 116 may cause output images corresponding to different input images to be simultaneously outputted to the display apparatus 3A and the display apparatus 3B illustrated in FIG. 1 via the display output interface unit 130 described later. Such a configuration enables the user to easily compare playing images by visually comparing the display apparatus 3A and the display apparatus 3B with each other, as in the example described with reference to FIG. 2, for example.

In addition, the output control section 116 may cause the output image acquired in a manner corresponding to each of the plurality of input images to be outputted to an identical apparatus simultaneously. For example, the output control section 116 may cause output images corresponding to different input images to be simultaneously outputted (transmitted) to the operation terminal 2 illustrated in FIG. 1 via the communication unit 120 described later. Such a configuration enables the user who operates the operation terminal 2 to simultaneously compare a plurality of output images on a single screen.

In addition, the output control section 116 may cause the output images corresponding to the different input images to be simultaneously outputted to each of the display apparatus 3A and the display apparatus 3B, and cause each of the output images to be simultaneously outputted to the operation terminal 2. Such a configuration enables the user who operates the operation terminal 2 to perform various operations while confirming the output images displayed on the display apparatus 3A and the display apparatus 3B on a screen of the operation terminal 2.

In order to be able to make comparison more easily, the output control section 116 may acquire an output image to reduce a difference among respective output images corresponding to a plurality of input images, as compared with a difference among the plurality of input images.

For example, the output control section 116 may acquire an output image and cause the output image to be outputted to reduce a difference in a format parameter. The output control section 116 does not need to reduce differences in all of format parameters acquired by the format acquisition section 112, but may acquire an output image to reduce a difference in at least one of the format parameters. The output control section 116 may acquire the format parameter either from the format acquisition section 112 or from the storage unit 150.

For example, the output control section 116 may perform processing, as for format parameters having a difference to be reduced, to allow the format parameters to be identical among a plurality of output images, to acquire an output image. The processing for causing the format parameters to be identical may be, for example, processing, in which any one input image of a plurality of input images is used as a reference to align a format parameter of an output image corresponding to another input image with a format parameter of the input image serving as the reference. Alternatively, processing may be adopted in which the format parameter of the output image corresponding to each of the input images may be aligned with a predetermined reference value.

For example, in a case where output images are obtained by performing processing to allow the frame rates to be identical, a frame included in an input image having a lower frame rate (less frames) is outputted a plurality of times in accordance with an input image having a higher frame rate (more frames) to thereby output an output image with increased number of frames. Alternatively, in such a case, a frame included in the input image having a higher frame rate may be thinned out and outputted in accordance with the input image having a lower frame rate, to thereby output an output image with reduced number of frames. In addition, it is possible to use a well-known image processing technique in performing processing to allow resolutions or aspect ratios to be identical.

Such a configuration makes it possible to reduce the difference in the image formats among output images, thus enabling the user to make the comparison more easily.

In addition, the output control section 116 may acquire an output image in a manner corresponding to each of the plurality of input images and may cause the output image to be outputted to reduce the difference in the subject parameter for the subjects included in each of the plurality of input images. The output control section 116 does not need to reduce the differences in all of subject parameters acquired by the image analysis section 114, but may acquire an output image to reduce a difference in at least one of the subject parameters. The output control section 116 may acquire the subject parameter either from the image analysis section 114 or from the storage unit 150.

For example, the output control section 116 may perform image processing, as for subject parameters having a difference to be reduced, to allow the subject parameters to be identical among a plurality of output images, to generate output images. The image processing for causing the subject parameters to be identical may be, for example, processing, in which any one input image of a plurality of input images is used as a reference to align a subject parameter of an output image corresponding to another input image with a subject parameter of the input image. Alternatively, processing may be adopted in which the subject parameter of the output image corresponding to each of the input images may be aligned with a predetermined reference value.

For example, in a case where an output image is obtained by performing processing to allow the dominant hand of the subject or the dominant foot of the subject to be identical, it may be judged whether or not left-right reversal processing is necessary for each input image to make alignment with any input image of a plurality of input images. An input image judged to be necessary may be subjected to the left-right reversal processing to generate an output image, while an input image judged to be unnecessary may be used as it is to be acquired as an output image.

The description has been given above of the case where the dominant hand of the subject and the dominant foot of the subject are each identical; however, likewise, also for other subject parameters, it is possible to acquire an output image to allow the subject parameters to be identical among output images, using a well-known image processing technique.

Such a configuration makes it possible to reduce the difference in appearances of the subject among output images, thus enabling the user to make the comparison more easily.

In addition, the output control section 116 may acquire an output image corresponding to each of the plurality of input images and may cause the output image to be outputted to reduce a difference (deviation) in timing. Here, the timing may be, for example, a motion timing of the subject. The output control section 116 may, for example, cause an output image to be outputted on the basis of a key frame specified in each of the plurality of input images and corresponding among the plurality of input images, in order to reduce the difference in timing.

The key frame corresponding among the plurality of input images may be, for example, a key frame in which the types of the key frames described above are identical. That is, in a case where the identical type of key frame has been specified among the plurality of input images, the output control section 116 may cause an output image to be outputted on the basis of the key frame. It is to be noted that the output control section 116 may acquire the key frame either from the image analysis section 114 or from the storage unit 150.

In addition, the output control section 116 may use corresponding key frames one by one to cause output images to be outputted, may use the key frames two by two to cause output images to be outputted, or may use more key frames to cause output images to be outputted, among the plurality of input images.

For example, in a case of using the corresponding key frames one by one to causes output images to be outputted among the plurality of input images, the output control section 116 may cause the output images to be synchronized and outputted using the key frame as a reference frame in each of the input images. For example, the output control section 116 may cause the output image to be outputted using the reference frame as a start frame. In a case of being applied to a playing image of sports, such a configuration allows output (display reproduction) of an output image to be started from a time point of starting a certain motion, thus enabling the user to compare motions more easily.

It is to be noted that, in a case where output images are outputted using the corresponding key frames one by one among the plurality of input images, each output image may be outputted, using, as a reference, an output image at which all frames are outputted first. For example, the output control section 116 may terminate the output at a time point when all the frames have been outputted for a certain output image, or may repeat the output in which the corresponding key frame is used as the start frame again, to allow loop reproduction to be performed. Such a configuration allows the output times of the output images to be identical, thus reducing a sense of discomfort of the user.

In addition, in a case where the output control section 116 causes output images to be outputted using the corresponding key frames two by two among the plurality of input images, in each of the input images, the two key frames may be used as a start frame and an end frame to cause the output images to be outputted. For example, among the two key frames, a key frame having a smaller frame number may be regarded as the start frame, and a key frame having a larger frame number may be regarded as the end frame. Then, the output control section 116 may perform speed adjustment of output (display reproduction) on the basis of the start frame and the output frame to cause the output images to be outputted. For example, the speed adjustment may be performed using, as a reference, the speed of any one input image of the plurality of input images to cause an output images corresponding to each of the plurality of input images to be outputted.

Such a configuration enables the user to make the comparison more easily regarding the timing. For example, in a case of being applied to a playing image of sports, such a configuration allows the start and the end of a certain motion to be aligned, thus making it possible to compare differences in forms, and the like more easily.

In addition, also in a case where more than two key frames are used to cause output images to be outputted in respective input images, the output control section 116 may perform key frame-based speed adjustment to cause the output images to be outputted, similarly to the example described above. However, in such a case, the output control section 116 may change a scale factor regarding the speed adjustment before and after the key frame during the output of outputted images.

Description is given below by exemplifying a case where three key frames of a first key frame, a second key frame, and a third key frame correspond among a plurality of input images. At this time, the output control section 116 may cause output images to be outputted by performing speed adjustment on the basis of the first key frame and the second key frame for a frame between the first key frame and the second key frame. Then, the output control section 116 may cause output images to be outputted by performing speed adjustment on the basis of the second key frame and the third key frame for a frame between the second key frame and the third key frame.

Such a configuration makes it possible to make comparison by aligning timings in more detail.

The above-described reduction in the difference may be performed on the basis of a reproduction condition set by an operation of the user who uses the operation terminal 2. The reproduction condition may correspond to, for example, a point to be compared by the user. For example, the output control section 116 may determine a difference to be reduced on the basis of the reproduction condition, and may cause an output image to be outputted to reduce the difference that is to be reduced.

For example, the output control section 116 may determine a parameter whose difference is reduced from among the format parameters on the basis of the reproduction condition. In addition, the output control section 116 may determine the parameter whose difference is reduced from among the subject parameters on the basis of the reproduction condition. In addition, the output control section 116 may determine the type and the number of key frames used to reduce the difference in timing on the basis of the reproduction condition.

Description is given below of the reproduction condition and several examples of the control of the output control section 116 according to the reproduction condition. However, the examples described below are merely exemplary, and the present technology is not limited to such examples.

For example, in a case where “confirmation of tennis swing” is set as the reproduction condition, the output control section 116 may reduce differences in those (make them the same) other than the aspect ratio among the format parameters while maintaining the aspect ratio in order to maintain a trajectory of the swing. Further, in such a case, the output control section 116 may reduce the differences in all of acquirable subject parameters (make them the same). Further, in such a case, the output control section 116 may perform speed adjustment on the basis of the three key frames of “take-back”, “impact”, and “follow-through” to cause output images to be outputted. Such a configuration reduces the difference in appearances of the subject and the difference in timing, thus making it possible to compare the motion of the body of the subject more easily.

In addition, in a case where “confirmation of hitting position of tennis ball” is set as the reproduction condition, the output control section 116 may reduce the difference in the positions of the racquet (an example of the subject) among the subject parameters. For example, the output control section 116 may perform image processing to generate an output image to allow the positions of the racquet to be identical in the “impact” key frame. In addition, in such a case, the output control section 116 may synchronize one key frame of the “impact” as the reference frame to cause an output image to be outputted. Such a configuration makes it possible to make comparison more easily as to where in the racquet the ball hits.

In addition, in a case where “confirmation of change in speed of tennis swing” is set as the reproduction condition, the output control section 116 may reduce differences in those (make them the same) other than the aspect ratio among the format parameters while maintaining the aspect ratio in order to maintain the trajectory of the swing. Further, in such a case, the output control section 116 may reduce the differences in all of acquirable subject parameters (make them the same). Further, in such a case, the output control section 116 may perform speed adjustment using the two key frames of the “take-back” and the “follow-through” as the start frame and the end frame, respectively, to cause output images to be outputted. Such a configuration makes it possible to compare changes in speed of the swing more easily while reducing the difference in appearances of the subject.

The communication unit 120 is a communication interface that mediates communication of the information processor 1 with other apparatuses. The communication unit 120 supports any wireless communication protocol or wired communication protocol, and establishes communication coupling with other apparatuses, e.g., via the communication network 5 described with reference to FIG. 1, or directly. For example, the communication unit 120 may transmit the output image to the operation terminal 2 under the control of the output control section 116 described above. In addition, the communication unit 120 may receive an image from the imaging apparatus 4. In addition, the communication unit 120 may receive information regarding operations of the user from the operation terminal 2.

The display output interface unit 130 is an interface that outputs an image to other apparatuses. For example, the display output interface unit 130 is coupled to the display apparatus 3A and the display apparatus 3B described with reference to FIG. 1, and outputs a different output image to each of the display apparatus 3A and the display apparatus 3B under the control of the output control section 116, for example.

The storage unit 150 stores a program, a parameter, and the like for the control unit 110 to execute each function. For example, the storage unit 150 may store one or a plurality of input images in advance. In addition, the storage unit 150 may store an image received by the communication unit 120 from the imaging apparatus 4 as an input image. In addition, the storage unit 150 may store a format parameter acquired by the format acquisition section 112 for each input image. In addition, the storage unit 150 may store a subject parameter acquired by the image analysis section 114 for each input image. In addition, the storage unit 150 may store information regarding a key frame specified by the image analysis section 114 for each input image, e.g., frame number and type.

In a case where the information regarding the format parameter, the subject parameter, and the key frame is acquired in advance for each input image and stored in the storage unit 150 in this manner, the output control section 116 is able to acquire the information from the storage unit 150. With such a configuration, a plurality of input images to be compared are selected, thus enabling the information processor 1 to cause output images to be outputted at a higher speed in a case where a reproduction condition is set.

### (Operation Terminal)

The description has been given above of the configuration example of the information processor 1. Next, description is given of a configuration example of the operation terminal 2 with reference to FIG. 4. FIG. 4 is a block diagram illustrating the configuration example of the operation terminal 2 according to the present embodiment. As illustrated in FIG. 4, the operation terminal 2 includes a control unit 210, a communication unit 220, an operation unit 230, a display unit 240, and a storage unit 250.

The control unit 210 functions as an arithmetic processor and a controller, and controls overall operations in the operation terminal 2 in accordance with various programs. For example, the control unit 210 causes the display unit 240 to display a screen for performing operations to allow the information processor 1 to output an output image desired by the user to the operation terminal 2 and the display apparatus 3. The user may operate a screen to be displayed on the display unit 240 by the control unit 210 to thereby select, from among a plurality of input images, a plurality of input images to be compared.

In addition, the user may operate a screen to be displayed on the display unit 240 by the control unit 210 to thereby set a reproduction condition in accordance with a point to be compared. It is to be noted that the user may make selection from among preset reproduction conditions prepared in advance, for example. The preset reproduction condition prepared in advance may be, for example, the above-mentioned “confirmation of tennis swing”, “confirmation of hitting position of tennis ball”, “confirmation of change in speed of tennis swing”, or the like. Alternatively, the user may be able to set the reproduction condition in more detail; for example, the user may be able to make selection, for the screen to be displayed on the display unit 240 by the control unit 210, as to whether or not each of the format parameters or each of the subject parameters is reduced (e.g., make them the same). In addition, the user may be able to select the type or the number of key frames to be synchronized among a plurality of output images for the screen to be displayed on the display unit 240 by the control unit 210.

In addition, the screen displayed on the display unit 240 by the control unit 210 may include a plurality of output images received by the communication unit 220 from the information processor 1. Such a configuration enables the user who operates the operation terminal 2 to easily make comparison by viewing a single screen. In addition, as described above, in a case where the information processor 1 outputs an output image to the display apparatus 3A, the display apparatus 3B, and the operation terminal 2 simultaneously, the user is able to confirm the output image displayed on the display apparatus 3A and the display apparatus 3B on the screen of the operation terminal 2.

It is to be noted that the screen displayed on the display unit 240 by the control unit 210 is not limited to such an example, and the control unit 210 may display variety of screens in cooperation with functions of the information processor 1; other examples are described later as modification examples.

In addition, the control unit 210 may control the communication unit 220 to transmit, to the information processor 1, information regarding operations of the user via the operation unit 230 described later. The information regarding operations of the user may be, for example, information regarding the above-described selection of a plurality of input images to be compared or information regarding the setting of the reproduction condition.

The communication unit 220 is a communication interface that mediates communication of the operation terminal 2 with other apparatuses. The communication unit 220 supports any wireless communication protocol or wired communication protocol, and establishes communication coupling with other apparatuses, e.g., via the communication network 5 described with reference to FIG. 1, or directly. For example, the communication unit 220 may transmit the information regarding operations of the user to the information processor 1 under the control of the control unit 210 described above. In addition, the communication unit 220 may receive an output image corresponding to each of the plurality of input images from the information processor 1.

The operation unit 230 accepts operations of the user. The operation unit 230 receives operations on various screens displayed on the display unit 240 by the control unit 210 described above. For example, the operation unit 230 may be implemented by a mouse, a keyboard, a touch sensor, a button, a switch, a lever, a dial, or the like.

The display unit 240 displays various screens under the control of the control unit 210 described above. It is to be noted that the operation unit 230 and the display unit 240 are each illustrated as a separate configuration in the example illustrated in FIG. 4; however, the operation terminal 2 may be provided with a touch panel display having both the function of the operation unit 230 and the function of the display unit 240.

The storage unit 250 stores data such as programs for the control unit 210 to execute respective functions, and parameters. For example, the storage unit 250 may store icons, and the like for the control unit 210 to cause the display unit 240 to display the various screens.

### 1-3. Operation Example

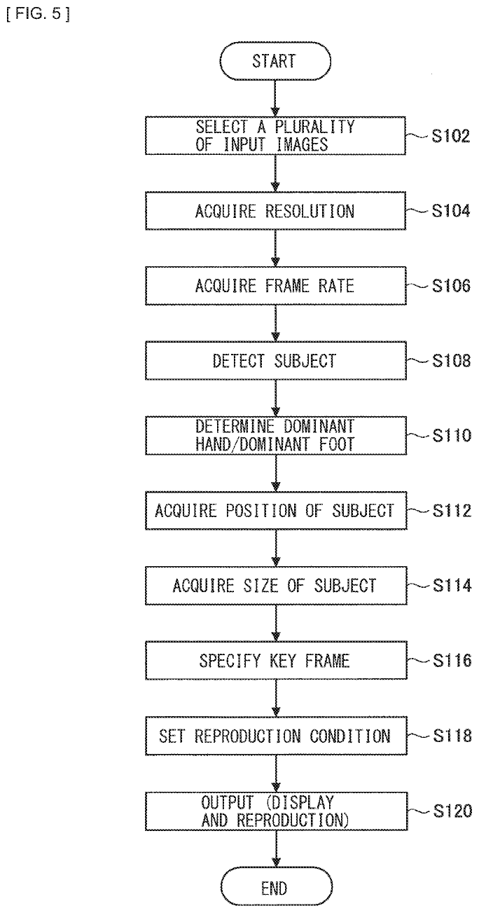

The description has been given above of the configuration examples of the information processor 1 and the operation terminal 2 according to the present embodiment. Next, description is given of an operation example of the present embodiment with reference to FIG. 5. FIG. 5 is a flowchart diagram illustrating an operation example of the present embodiment.

First, in step S102, an operation of the user is performed using the operation terminal 2 to select a plurality of input images to be compared from among input images stored in the storage unit 150 of the information processor 1. Each processing in the subsequent steps S104 to S116 may be performed independently in each of the plurality of input images selected in step S102.

In step S104, the format acquisition section 112 of the information processor 1 acquires resolution of each of the input images. In step S104, aspect ratios (an example of the format parameter) of the respective input images may be acquired simultaneously. In the subsequent step S104, the format acquisition section 112 acquires frame rates (an example of the format parameter) of the respective input images.

In the subsequent step S108, the image analysis section 114 of the information processor 1 detects a subject included in each of the input images. In the subsequent step S110, the image analysis section 114 determines the dominant hand or the dominant foot (an example of the subject parameter) of the subject detected in step S108. In the subsequent step S112, the image analysis section 114 acquires a position of the subject (an example of the subject parameter) detected in step S108. Further, in the subsequent step S114, the image analysis section 114 acquires a size of the subject (an example of the subject parameter) detected in step S108.

In the subsequent step S116, the image analysis section 114 specifies a key frame in each of the input images.

In the subsequent step S118, an operation of the user is performed using the operation terminal 2 to set a reproduction condition.

In the subsequent step S120, the output control section 116 of the information processor 1 acquires an output image as described above on the basis of the reproduction condition set in step S118, and causes the display apparatus 3 to output (display and reproduce) the output image.

The description has been given above of the operation example of the present embodiment. It is to be noted that, those illustrated in FIG. 5 are merely exemplary, and the operation of the present embodiment is not limited to such an example. For example, steps do not necessarily need to be processed in chronological order in the order illustrated in FIG. 5; the steps either may be processed in an order different from the order illustrated in FIG. 5, or may be processed in parallel.

In addition, in the example illustrated in FIG. 5, an example is illustrated, in which only the dominant hand of the subject, the dominant foot of the subject, the position of the subject, and the size of the subject are acquired as the subject parameters; however, another subject parameter may be acquired.

In addition, in the example illustrated in FIG. 5, an example is illustrated, in which the plurality of input images to be compared are selected in step S102 and thereafter a series of processing in steps S104 to S116 is performed; however, the present embodiment is not limited to such an example. As described above, the series of processing in steps S104 to S116 may be performed in advance for the input images stored in the storage unit 150 of the information processor 1, and the format parameter, the subject parameter, and the key frame may be stored in the storage unit 150. In a case where the series of processing in steps S104 to S116 has been performed in advance for the plurality of input images selected in step S102 in this manner, the series of processing in steps S104 to S116 may be skipped after step S102, and processing may proceed to the subsequent step S118.

### 1-4. Modification Examples

The description has been given above of the first embodiment of the present disclosure. Description is given below of several modification examples of the present embodiment. It is to be noted that each modification example described below may be applied to the present embodiment alone or in combinations. In addition, each modification example may be applied instead of the configuration described in the present embodiment, or may be additionally applied to the configuration described in the present embodiment.

### Modification Example 1

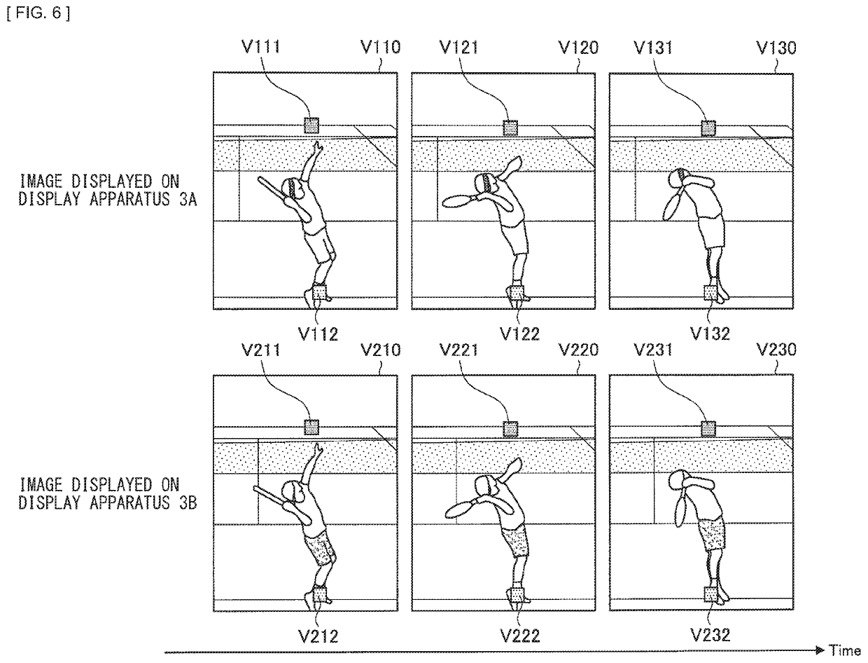

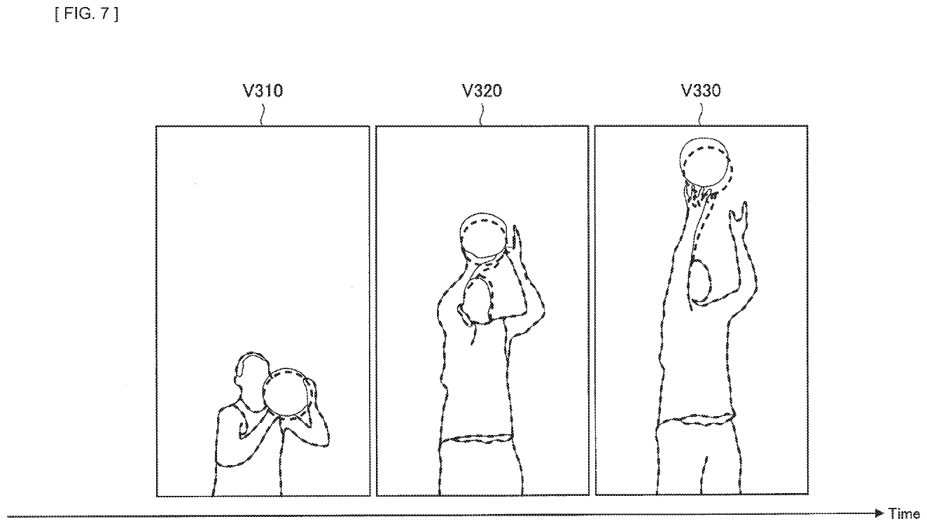

The output control section 116 may superpose a result of the image analysis performed by the image analysis section 114 on an output image to cause the output image to be outputted. For example, the output control section 116 may superpose a mark indicating the position of the subject obtained by the image analysis performed by the image analysis section 114 on the output image for outputting. Such an example is described, as Modification Example 1, with reference to FIG. 6. FIG. 6 is an explanatory diagram that describes Modification Example 1 according to the present embodiment.

As illustrated in FIG. 6, the output control section 116 causes output images V110, V120, and V130 to be outputted in order (in chronological order) to the display apparatus 3A and to be displayed. In addition, the output control section 116 simultaneously causes output images V210, V220, and V230 to be outputted in order (in chronological order) to the display apparatus 3B and to be displayed. It is to be noted that, in the example illustrated in FIG. 6, the output images V110, V120, and V130 and the output images V210, V220, and V230 correspond each other, respectively, and are displayed at the same time.

In the example illustrated in FIG. 6, marks V111, V121, V131, V211, V221, and V231 each indicating a position of a tennis ball as a subject are superposed, respectively, on the output images V110, V120, V130, V210, V220, and V230. In addition, in the example illustrated in FIG. 6, marks V112, V122, V132, V212, V222, and V232 each indicating a foot position of a person as a subject are superposed, respectively, on the output images V110, V120, V130, V210, V220, and V230.

Displaying the position of the subject in a superposed manner on the output image enables the user to grasp the position of the subject more easily. For example, in a case where a reproduction condition is so set as to reduce the difference in the position of the subject, the position of the subject is displayed in a superposed manner on the output image, thus enabling the user to confirm that the difference is correctly reduced.

### Modification Example 2

The output control section 116 may output a plurality of output images in a superimposed manner. For example, the output control section 116 may acquire an output image for each of the plurality of input images, superimpose the acquired plurality of output images on each other, and output them to the operation terminal 2 or the display apparatus 3. Then, the operation terminal 2 or the display apparatus 3 may display an image in which the plurality of output images are superimposed on each other. Such an example is described, as Modification Example 2, with reference to FIG. 7. FIG. 7 is an explanatory diagram that describes Modification Example 2 according to the present embodiment.

FIG. 7 illustrates images V310, V320, and V330 outputted by the output control section 116 by superimposing two output images on each other. The output control section 116 outputs the images V310, V320, and V330 in order (in chronological order).

The output control section 116 may cause a plurality of output image representations to be different from one another for superimposition. For example, in the example illustrated in FIG. 7, the superposition is performed to cause line types to differ for respective output images. It is to be noted that the present modification example is not limited to such an example; the output control section 116 may cause colors to differ for respective output images for the superimposition.

Such a configuration makes it possible to compare, for example, motions or forms of the subjects included in respective input images in more detail.

In addition, the output control section 116 may cause colors of respective regions to differ in accordance with magnitude of a difference among the plurality of output images. For example, the output control section 116 may cause an image to be outputted, in which a region having a larger difference among the plurality of output images has a color closer to red. Such a configuration enables the user to more easily grasp a part having a large difference in the motion or the form of the subject, for example.

### Modification Example 3

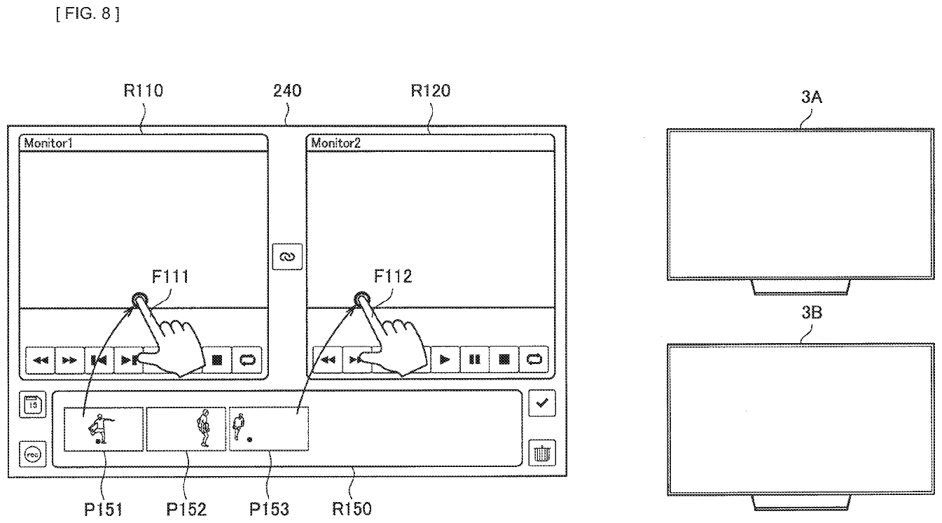

The description has been given, in the foregoing embodiment, of the case where the control unit 110 of the information processor 1 automatically specifies a key frame; however, the key frame may be specified on the basis of operations of the user who uses the operation terminal 2. Such an example is described, as Modification Example 3, with reference to FIG. 8 to FIG. 10. FIG. 8 to FIG. 10 are each an explanatory diagram that describes Modification Example 3 according to the present embodiment.

In a state illustrated in FIG. 8, input images to be compared are not selected; nothing is displayed on the display apparatus 3A and on the display apparatus 3B. Now, description is given, with reference to FIG. 8, of an example of operations of the user for selecting a plurality of input images to be compared, which are performed on screens displayed on the display unit 240 of the operation terminal 2.

In a thumbnail image display region R150 of the screen displayed on the display unit 240, thumbnail images P151 to P153 indicating respective input images stored in the storage unit 150 of the information processor 1 are displayed. The user confirms the thumbnail images P151 to P153, and moves the thumbnail images indicating input images desired to be compared to a first preview region R110 corresponding to the display apparatus 3A or to a second preview region R120 corresponding to the display apparatus 3B. An operation for such movement may be a so-called drag and drop operation.

In the example illustrated in FIG. 8, a user's finger F111 has moved the thumbnail image P151 to the first preview region R110, and a user's finger F112 has moved the thumbnail image P153 to the second preview region R120. These operations enables the user to select, as input images to be compared, an input image corresponding to the thumbnail image P151 and an input image corresponding to the thumbnail image P153. It is to be noted that, in the following description of the present modification example, an output image outputted from the information processor 1 in a manner corresponding to the input image corresponding to the thumbnail image P151 is referred to as a first input image, while the input image corresponding to the thumbnail image P153 is referred to as a second input image. In addition, in the following description of the present modification example, an output image corresponding to the first input image is referred to as a first output image, while an output image corresponding to the second input image is referred to as a second output image.

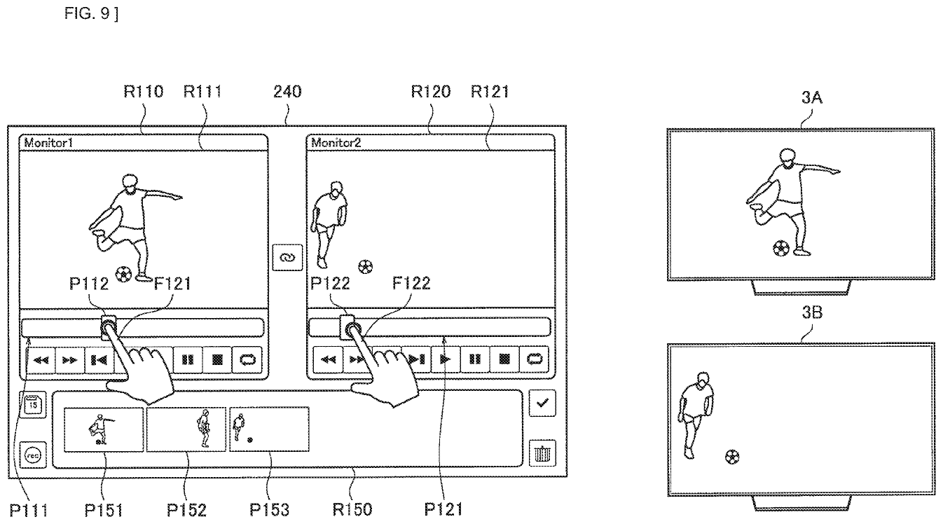

As a result of the above-described operations, the first input image and the second input image are displayed, respectively, on the display apparatus 3A and the display apparatus 3B as illustrated in FIG. 9. In addition, input images are also displayed on the display unit 240. In the example illustrated in FIG. 9, the first input image and the second input image are displayed, respectively, in a first preview image display region R111 of the first preview region R110 and a second preview image display region R121 of the second preview region R120.

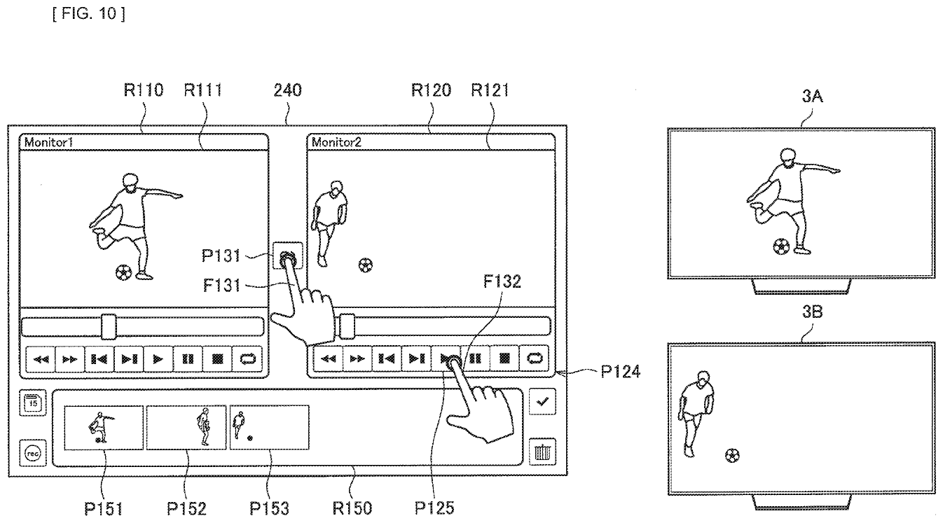

Now, description is given, with reference to FIG. 9 and FIG. 10, of operations of the user for specifying corresponding key frames among input images, which are performed on the screens displayed on the display unit 240 of the operation terminal 2.

In order to specify a key frame in the first input image, the user moves a slider P112 of a reproduction bar P111 included in the first preview region R110 to allow a desired frame (key frame) to be displayed in the second preview image display region R121. In the example illustrated in FIG. 9, such operations are illustrated using a user's finger F121. Likewise, in order to specify a key frame in the second input image, the user moves a slider P122 of a reproduction bar P121 included in the second preview region R120 to allow a desired frame (key frame) to be displayed in the second preview image display region R121. In the example illustrated in FIG. 9, such operations are illustrated using a user's finger F122. At this time, information regarding movement of each slider is transmitted from the operation terminal 2 to the information processor 1, and the information processor 1 changes the frame of output image to be outputted in accordance with the movement of each slider.

When a key frame is displayed in each input image as described above, the user presses a link button P131 for associating the key frame of the first input image and the key frame of the second input image with each other as indicated by a finger F131 illustrated in FIG. 10. When the link button P 131 is pressed, the key frames specified as described with reference to FIG. 9 are associated between the first input image and the third input image, and synchronized in the reproduction or the like. For example, at this time, information regarding the association of the key frame may be transmitted from the operation terminal 2 to the information processor 1.

For example, after the link button P131 is pressed, when pressing a reproduction start button P125 included in a reproduction control button group P124 displayed in the second preview region R120 as indicated by a finger F132 illustrated in FIG. 10, the two input image are reproduced in synchronization with each other.

### Modification Example 4

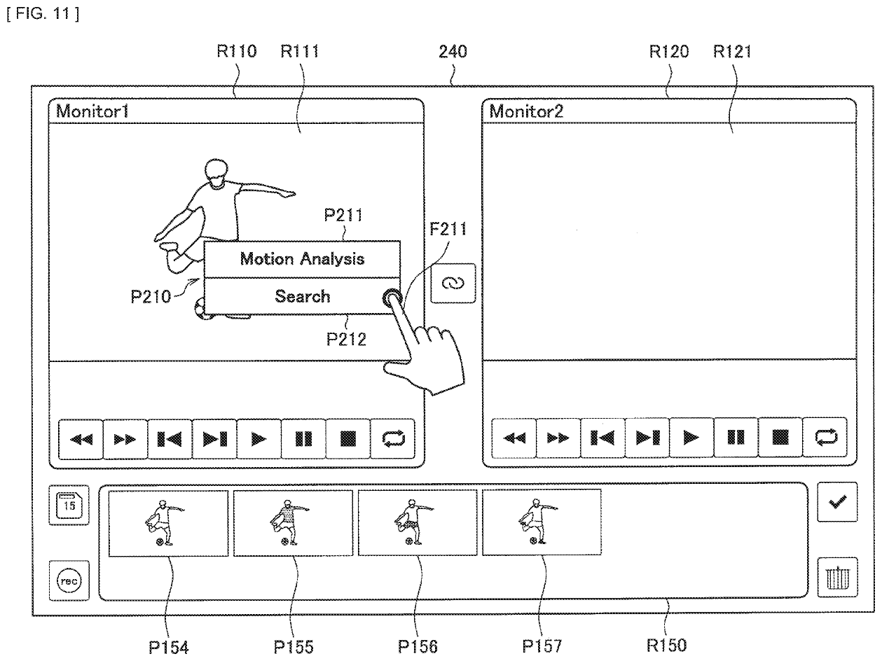

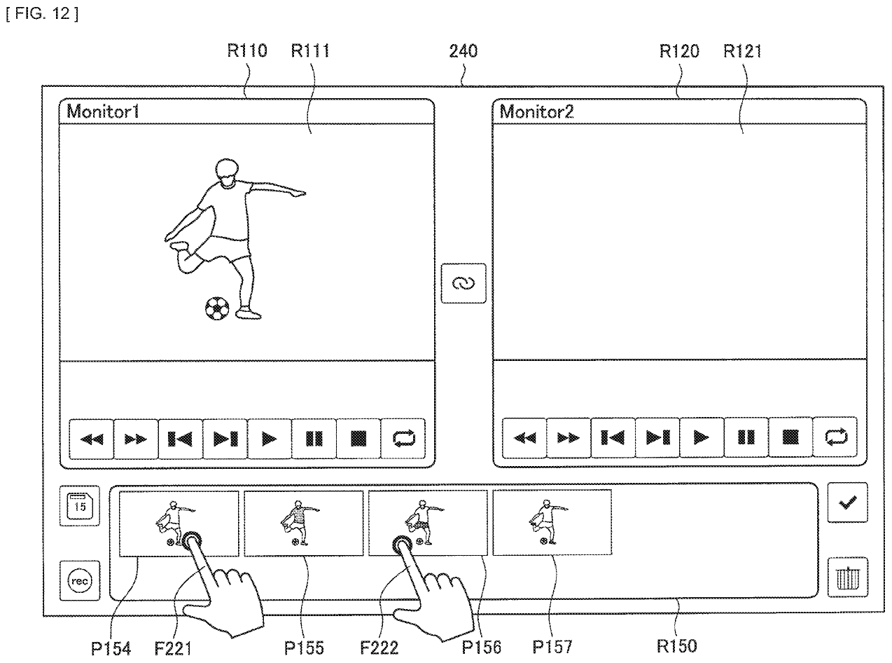

The control unit 110 of the operation terminal 2 may cause more varieties of screens to be displayed on the display unit 240. For example, in order to select a plurality of input images to be compared, the control unit 110 of the operation terminal 2 may display a screen that provides a function of searching similar images. Such an example is described, as Modification Example 4, with reference to FIG. 11 and FIG. 12. FIG. 11 and FIG. 12 are each an explanatory diagram that describes Modification Example 4 according to the present embodiment.

In the example illustrated in FIG. 11, a menu button group P210 is displayed on the display unit 240 of the operation terminal 2. In a case where the operation unit 230 of the operation terminal 2 is a touch panel, the menu button group P210 may be displayed by a long press (touch for a predetermined period of time or longer), for example.

When a button P212 for searching of similar images, among a button P211 and the button P212 included in the menu button group P210, is pressed as indicated by a finger F211 illustrated in FIG. 11, the searching of similar image is performed. Such searching of similar images may be performed by the control unit 110 of the information processor 1, for example, and may be processing of searching a frame similar to a frame (hereinafter, referred to as a search target frame) displayed in the first preview image display region R111 from among the input images stored in the storage unit 150 of the information processor 1.

In the example illustrated in FIG. 11, thumbnail images P154 to P157 indicating input images obtained as a result of the searching of similar images are displayed in the thumbnail image display region R150. It is to be noted that the thumbnail images displayed in the thumbnail image display region R150 as a result of the searching of similar images may be images corresponding to frames determined to be similar to the search target frame in the searching of similar images in each input image. In addition, the thumbnail images displayed in the thumbnail image display region R150 may include, for example, thumbnail images indicating different frames of the identical input image.

Next, as illustrated in FIG. 12, the user selects a plurality of input images to be compared. In the example illustrated in FIG. 12, as input images to be compared, an input image corresponding to the thumbnail image P154 is selected by a finger F221, and an input image corresponding to the thumbnail image P156 is selected by a finger F222. It is to be noted that the selected input images may be associated with each other using the frames indicated by the respective thumbnail images as key frames.

According to Modification Example 4 described above, the user is able to select a plurality of input images to be compared more easily, which is particularly effective in a case where there are many input images stored in the storage unit 150 of the information processor 1. In addition, it is also possible to specify a corresponding key frame among input images by searching of similar images, thus making it also possible to omit the operations for specifying the key frame.

### Modification Example 5

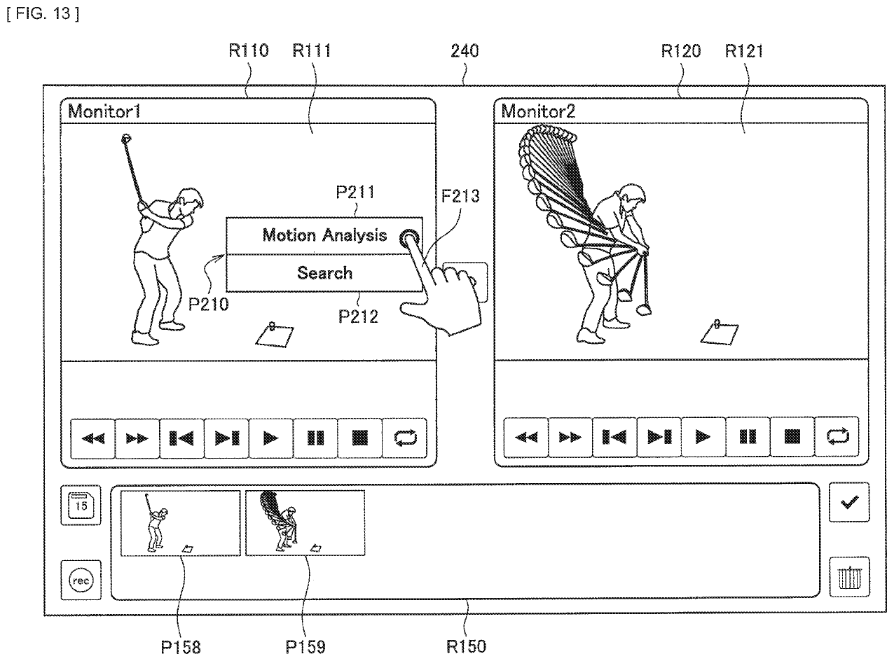

In addition, the control unit 110 of the operation terminal 2 may cause the image analysis section 114 of the information processor 1 to analyze an input image to display a screen for presenting a result of such analysis. Such an example is described, as Modification Example 5, with reference to FIG. 13. FIG. 13 is an explanatory diagram that describes Modification Example 5 according to the present embodiment.

When the button P211 for motion analysis of the subject is pressed as indicated by a finger F213 illustrated in FIG. 13, the motion analysis is performed by the image analysis section 114 of the information processor 1. Such motion analysis may be directed to, for example, an image displayed in the first preview image display region R111. It is to be noted that, in FIG. 13, the image displayed in the first preview image display region R111 is an input image corresponding to a thumbnail image P158 displayed on the thumbnail image display region R150.

When the motion analysis is performed, as illustrated in FIG. 13, an image indicating a motion analysis result is displayed in the second preview image display region R121, and a thumbnail image corresponding to the image indicating the motion analysis result is displayed in the thumbnail image display region R150. As illustrated in FIG. 13, the image indicating the motion analysis result may be an image indicating a trajectory of the motion of the subject in a superimposed manner, and may be a still image or a moving image.

According to Modification Example 5 described above, it becomes possible for the user of the operation terminal 2 to cause the image analysis function of the information processor 1 to be executed and to confirm the analysis result on the screen displayed on the display unit 240 of the operation terminal 2. For example, in the example illustrated in FIG. 13, it is possible to grasp a form, a change in speed, and the like of golf swing more easily.

### Modification Example 6

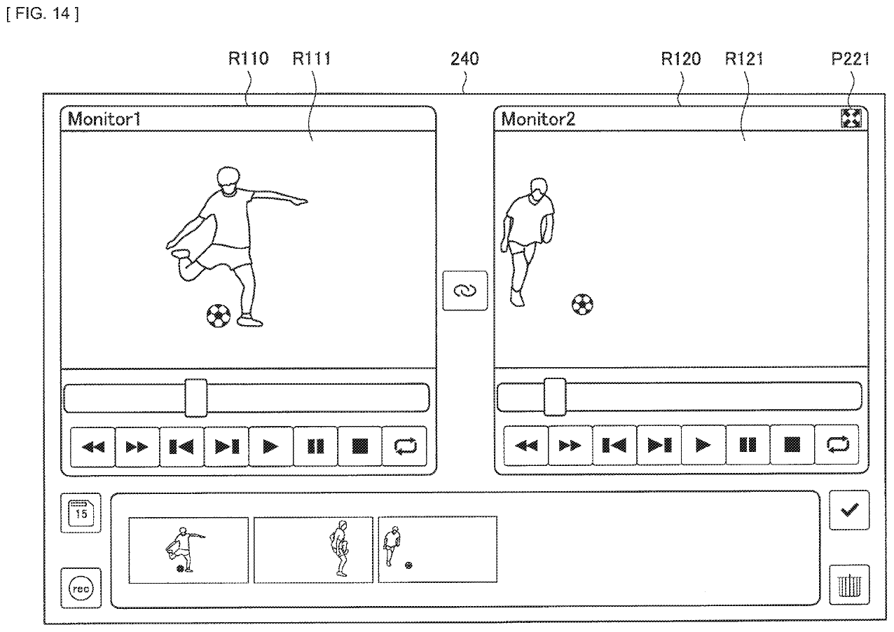

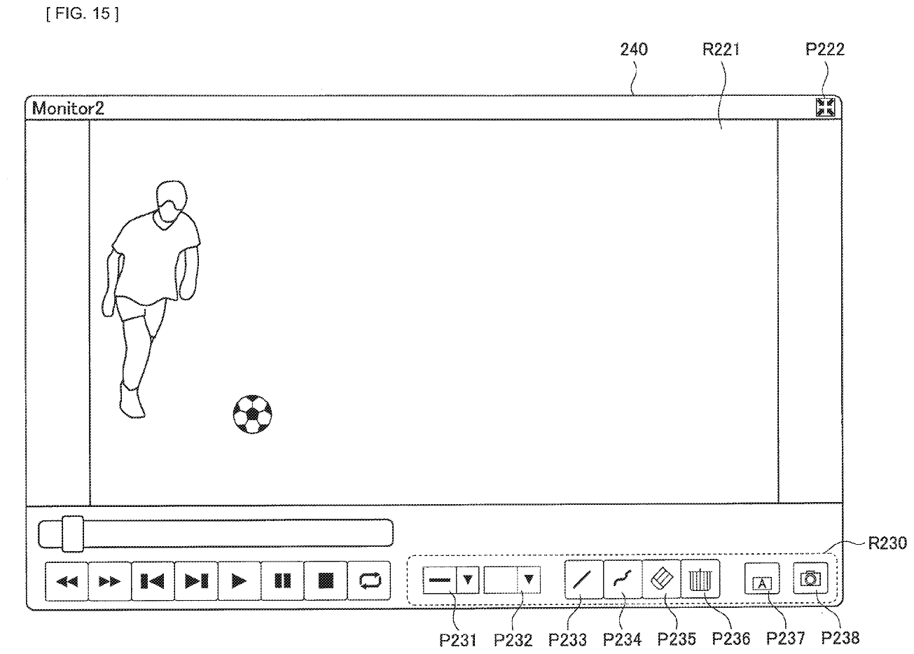

In addition, the control unit 110 of the operation terminal 2 may display a screen for performing an operation of adding (rendering) an annotation to an output image outputted by the output control section 116 of the information processor 1. Such an example is described, as Modification Example 6, with reference to FIG. 14 and FIG. 15. FIG. 14 and FIG. 15 are each an explanatory diagram that describes Modification Example 6 according to the present embodiment.

In the example illustrated in FIG. 14, an enlargement button P221 is displayed in the second preview region R120. When such an enlargement button P221 is pressed, a screen as illustrated in FIG. 15 is displayed on the display unit 240.

The screen of the display unit 240 illustrated in FIG. 15 is larger than the second preview region R120 illustrated in FIG. 14, and includes an enlarged preview display region R221 in which the second preview region R120 illustrated in FIG. 14 is displayed in an enlarged manner.

In addition, the screen of the display unit 240 illustrated in FIG. 15 includes an annotation menu bar R230. In the annotation menu bar R230, pull-down lists P231 and P232 and buttons P233 to P238 are displayed. The pull-down lists P231 and P232 and the buttons P233 to P238 can be used to render an annotation into the enlarged preview display region R221. It is to be noted that the annotation rendered into the enlarged preview display region R221 may be rendered in a similar manner to an output image displayed on the display apparatus 3B.

The pull-down list P231 is a pull-down list for selecting thickness of a line to be rendered. In addition, the pull-down list P232 is a pull-down list for selecting a color of the line to be rendered. In addition, a button P233 is a button to be selected in rendering a straight line. In addition, a button P234 is a button to be selected in rendering a free line (line by freehand). In addition, a button P235 is a button to be selected in using an eraser that partially erases a rendering content. In addition, a button P236 is a button to be selected in clearing the rendering content at once. In addition, a button P237 is a button for switching between display and non-display of the rendering content in the enlarged preview display region R221. In addition, a button P238 is a button for saving a snapshot of an image displayed on the enlarged preview display region R221.

In addition, in the example illustrated in FIG. 15, the screen displayed on the display unit 240 includes a reduction button P222 for returning to the screen illustrated in FIG. 14. For example, the user may press the reduction button P222 in a case where the rendering of the annotation is completed; when the reduction button P222 is pressed, the annotation rendered on the screen illustrated in FIG. 15 is also displayed in the second preview region R120 illustrated in FIG. 14.

It is to be noted that the description has been given above of the rendering of the annotation for the output image displayed on the display apparatus 3B; however, the rendering of the annotation may also be possible similarly for the output image displayed on the display apparatus 3A. In addition, rendering of an annotation on one output image may allow for rendering of the identical annotation on a corresponding position of the other output image.

### 1-5. Effects

The description has been given above of the first embodiment of the present disclosure. According to the first embodiment of the present disclosure, it becomes possible to make comparison more easily among a plurality of images.

### 2. Second Embodiment

The description has been given above of the first embodiment of the present disclosure. Next, description is given of a second embodiment of the present disclosure.

### 2-1. Overview

For example, in order to relay sports, images obtained by imaging of sports plays have been edited. The editing work has included, for example, a work of selecting an important image for each scene from among images captured by a plurality of cameras, and a work of extracting and enlarging an important part from the images. Such an image editing work has often involved manual labors and has a large human burden. Therefore, the second embodiment of the present disclosure described below proposes an information processor, an information processing method, and a program that make it possible to reduce the human burden associated with such editing of images.

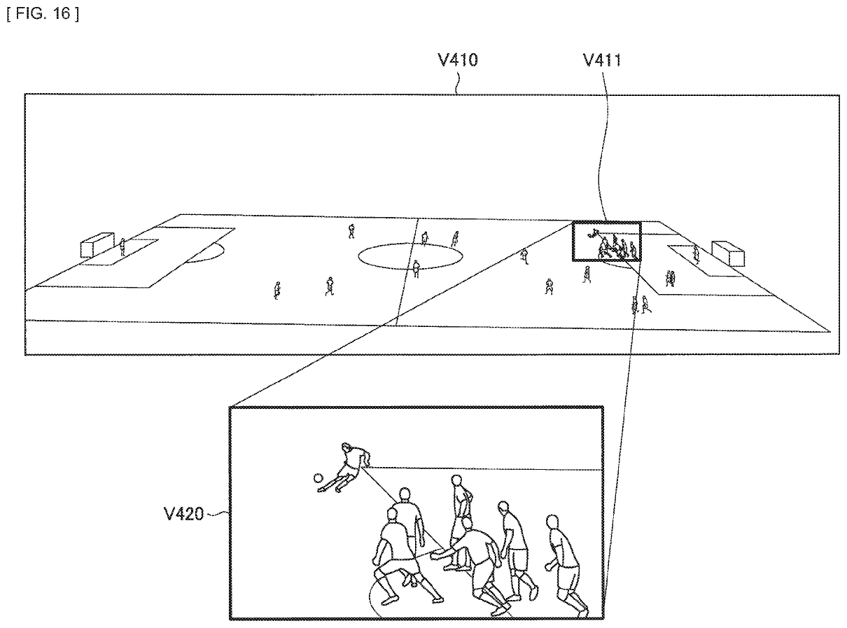

FIG. 16 is an image diagram that describes an overview of the second embodiment of the present disclosure. In the present embodiment, for example, a certain region is extracted from an input image obtained by imaging of sports plays or the like, and the extracted region is displayed in an enlarged manner. Hereinafter, the region extracted from the input image is referred to as an extracted region.

FIG. 16 illustrates an input image V410 so obtained by imaging of soccer plays as to include the entire soccer field. In the present embodiment, the input image is desirably a high-resolution image obtained by imaging of a wide range; for example, the input image may be a panoramic image obtained by synthesizing images obtained by imaging of a plurality of imaging apparatuses. However, the input image according to the present embodiment is not limited to such an example.

In the example illustrated in FIG. 16, an extracted region V411 is extracted from the input image V410. The extracted region V411 may be an important region of the input image V410; in the example illustrated in FIG. 16, a region where players are densely clustered and a soccer ball is included is extracted as the extracted region V411.

An enlarged image V420 obtained by enlarging such an extracted region V411 is displayed as an output image, thereby making it possible to grasp a status more easily than a case where the entire input image V410 is displayed. In addition, the above processing is automatically performed, thereby largely reducing the human burden associated with the editing work of images.

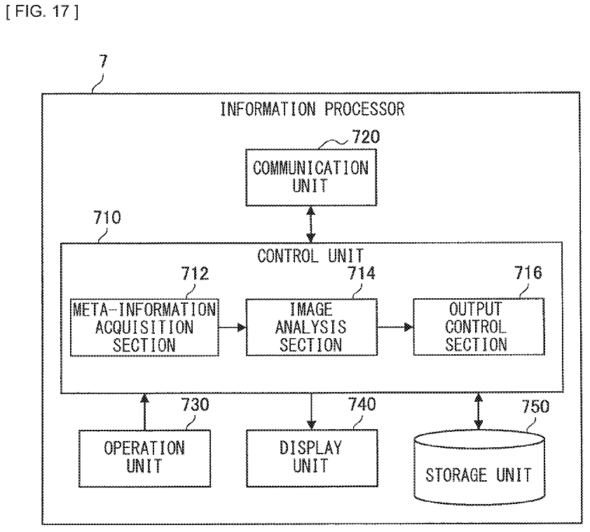

Hereinafter, description is given of a configuration example of the present embodiment for achieving such effects with reference to FIG. 17.

### 2-2. Configuration Example

FIG. 17 is a block diagram illustrating a configuration example of an information processor 7 according to the present embodiment. As illustrated in FIG. 17, the information processor 7 includes a control unit 710, a communication unit 720, an operation unit 730, a display unit 740, and a storage unit 750.

The control unit 710 functions as an arithmetic processor and a controller, and controls overall operations in the information processor 7 in accordance with various programs. In addition, the control unit 710 according to the present embodiment has functions as a meta-information acquisition section 712, an image analysis section 714, and an output control section 716 as illustrated in FIG. 17.

The meta-information acquisition section 712 acquires meta-information regarding input images. The meta-information obtained by the meta-information acquisition section 712 may include event occurrence information regarding an event that has occurred in the input image and subject information regarding the subject.

In a case where the input image was obtained by imaging of a soccer game, the event occurrence information may include, for example, information regarding shots (an example of the event that has occurred in the input image). The information regarding shots may include, for example, the number of shots, time when the shot was made, team or uniform number of a player who made the shot, whether or not the shot was scored, and the like.

In addition, in a case where the input image is obtained by imaging of a soccer game, the subject information may include, for example, information on a position, etc. of each player (an example of the subject) or a soccer ball (an example of the subject) at each time.

The meta-information acquisition section 712 may acquire (receive) meta-information from other apparatuses via the communication unit 720. For example, in a professional sports game, there may be an organization that provides the above-described meta-information in some cases; the meta-information acquisition section 712 may acquire the meta-information from apparatuses of such an organization.

However, the creation of the meta-information is costly, and thus it may be difficult for a school and a general sports club to be sufficiently supplied with the above-described meta-information in some cases. Even in such a case, when only a portion of the above-described meta-information (e.g., only the number of shots or the time when the shot was made, etc.) is provided, the meta-information acquisition section 712 may receive only such a portion of the meta-information from the other apparatus.

In addition, the meta-information acquisition section 712 causes the display unit 740 to display a screen for inputting meta-information, and may acquire the meta-information on the basis of the operations of the user accepted by the operation unit 730. However, such operations increases the human burden, and thus the meta-information to be acquired on the basis of operations of a user is desirably less.

Therefore, the image analysis section 714 acquires meta-information not included in the meta-information acquired by the meta-information acquisition section 712 by analysis of the input image for complement. Such a configuration makes it possible to reduce the human burden associated with the acquisition of the meta-information.

For example, in a case where the meta-information includes only the information on the time of the shot, which is an example of the event occurrence information, the image analysis section 714 may acquire information on the position of players and the ball (examples of the subject information) by analysis of the input images.

In addition, the image analysis section 714 may utilize the meta-information obtained by the meta-information acquisition section 712 in the analysis of the input images. For example, the image analysis section 714 may analyze the input images on the basis of the input images and the meta-information acquired by the meta-information acquisition section 712, and may acquire meta-information not included in the meta-information obtained by the meta-information acquisition section 712. For example, in a case where the meta-information includes only the information on the time of the shot, which is an example of the event occurrence information, the image analysis section 714 may specify information on the team who made the shot, on the basis of the information on the positions of players and the ball (examples of the subject information) obtained by the analysis of the input images.

Such a configuration makes it possible, for example, to limit the frame to be analyzed on the basis of the meta-information acquired by the meta-information acquisition section 712, thus making it possible to reduce processing load associated with the analysis of the input images.

It is to be noted that the description has been given above, as an example, of the case where the meta-information includes only the information on the time of the shot, which is an example of the event occurrence information; however, of course, the present embodiment is not limited to such an example, and is also applicable to another example.

For example, in a case where the meta-information includes information on the position of a certain player, which is an example of the subject information, but lacks (does not include) the information on the uniform numbers of respective players or the team, the image analysis section 714 may acquire such information on the uniform number or the team by analysis of the input images. For example, the image analysis section 714 may acquire the information on the uniform number or the team by recognizing, on the basis of the information on positions of players, numerals of the uniform number of the players existing at respective positions or by recognizing the team from the color of the uniform.

In this manner, the image analysis section 714 may acquire the lacking information by analysis of the input images in accordance with the information acquired by the meta-information acquisition section 712.

Further, the image analysis section 714 may specify an extracted region on the basis of the meta-information acquired by the meta-information acquisition section 712 and the meta-information acquired by the analysis of the input images. For example, as described above, in a case where the information on the team who made the shot is specified, it becomes possible to determine which team's goal area should be specified as the extracted region.

The output control section 716 causes the display unit 740 to output (display) an output image on the basis of the extracted region specified by the image analysis section 714. For example, the output control section 716 may extract an extracted region from the input image, perform enlargement processing on the extracted region in accordance with resolution of the extracted region and resolution of the display unit 740 to generate an output image, and cause the display unit 740 to display the output image.

It is to be noted that the output control section 716 may cause the display unit 740 to display the extracted region extracted from the input image as it is as an output image without being enlarged. Alternatively, in a case where the resolution of the display unit 740 is smaller than the resolution of the extracted region, the output control section 716 may perform reduction processing on the extracted region in accordance with the resolution of the extracted region and the resolution of the display unit 740 to generate an output image, and cause the display unit 740 to display the output image.

The communication unit 720 is a communication interface that mediates communication of the information processor 7 with other apparatuses. The communication unit 720 supports any wireless communication protocol or wired communication protocol, and establishes communication coupling with unillustrated other apparatuses through an unillustrated communication network, or directly. For example, the communication unit 720 may receive meta-information from other apparatuses under the control of the meta-information acquisition section 712.

The operation unit 730 accepts operations of a user. For example, the operation unit 730 may be implemented by a mouse, a keyboard, a touch sensor, a button, a switch, a lever, a dial, or the like.

The display unit 740 displays under the control of the control unit 710. For example, the display unit 740 displays and reproduces an output image on the basis of an extracted region under the control of the output control section 716 described above.

The storage unit 750 stores data such as programs for the control unit 710 to execute respective functions, and parameters. For example, the storage unit 750 may store input images, meta-information received by the communication unit 720 from other apparatuses, and the like.

### 2-3. Operation Example

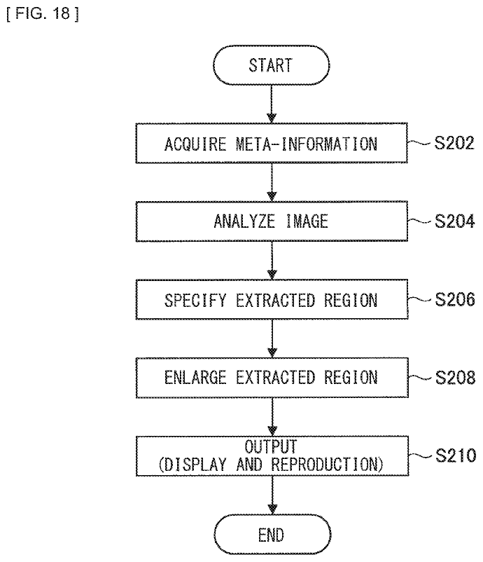

The description has been given above of the configuration example of the information processor 7 according to the present embodiment. Next, description is given of an operation example of the present embodiment with reference to FIG. 5. FIG. 18 is a flowchart diagram illustrating an operation example of the present embodiment.

First, in step S202, the meta-information acquisition section 712 acquires meta-information. The meta-information acquired in step S202 may not include enough meta-information to specify an extracted region, and may lack necessary meta-information to specify the extracted region.

Next, in step S204, the image analysis section 714 acquires meta-information by analysis of the input images. The meta-information acquired in step S204 may be, for example, lacking meta-information that is not included in the meta-information acquired by the meta-information acquisition section 712, among the necessary meta-information to specify the extracted region.

Next, in step S206, the image analysis section 714 specifies the extracted region on the basis of the meta-information acquired by the meta-information acquisition section 712 and the meta-information acquired by the analysis of the input images.

Next, in step S208, the output control section 716 enlarges the extracted region to generate an output image. Then, in the subsequent step S210, the output control section 716 causes the display unit 740 to output (display and reproduce) the output image.

### 2-4. Modification Example

The description has been given above of the second embodiment of the present disclosure. Hereinafter, description is given of a modification example of the present embodiment. It is to be noted that the modification example described below either may be applied instead of the configuration described in the present embodiment, or may be additionally applied to the configuration described in the present embodiment.

As described above, the meta-information acquisition section 712 may acquire the meta-information from other apparatuses. However, time information of the meta-information provided from other apparatuses may not coincide with time information of the input image stored in the storage unit 750 of the information processor 7 in some cases.

Therefore, the image analysis section 714 may adjust the time information of the input image and the time information of the meta-information acquired by the meta-information acquisition section 712 by analysis of the input images. For example, the image analysis section 714 collates an event (e.g., a shot, etc.) recognized by the analysis of the input images and an event included in the meta-information acquired by the meta-information acquisition section 712 with each other to thereby automatically perform such an adjustment.

### 2-5. Effects and Supplementals

The description has been given above of the second embodiment of the present disclosure. According to the second embodiment of the present disclosure, it is possible to reduce the human burden associated with the editing of images.

It is to be noted that the second embodiment of the present disclosure described above can also be combined with the first embodiment of the present disclosure.

### 3. Application Example

The technology according to an embodiment of the present disclosure can be applied to a variety of products. For example, the technology according to an embodiment of the present disclosure may be applied to a surgery room system.

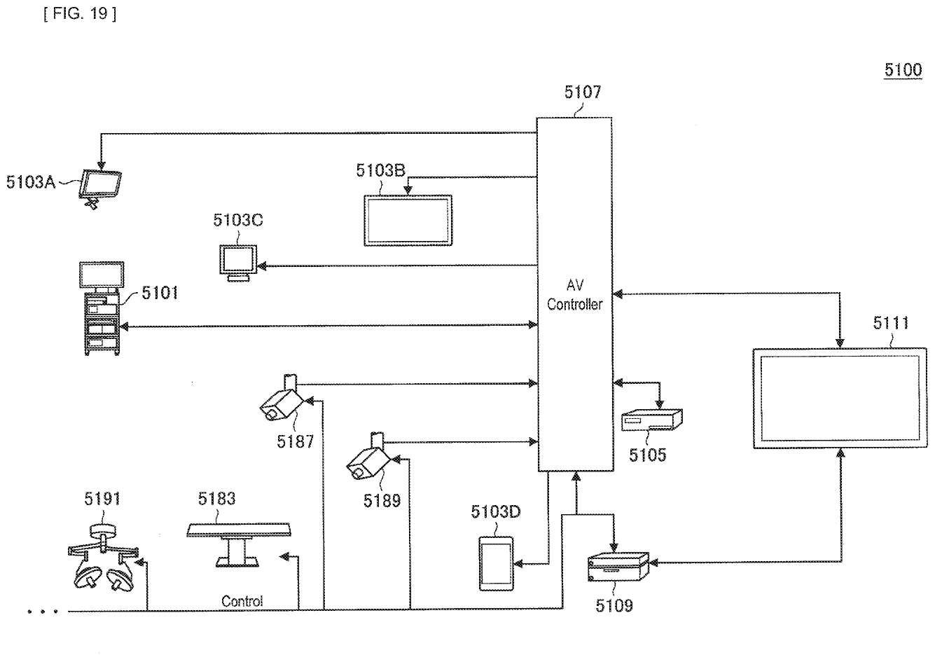

FIG. 19 is a view schematically depicting a general configuration of a surgery room system 5100 to which the technology according to an embodiment of the present disclosure can be applied. Referring to FIG. 19, the surgery room system 5100 is configured such that a group of apparatus installed in a surgery room are connected for cooperation with each other through an audiovisual (AV) controller 5107 and a surgery room controlling apparatus 5109.

In the surgery room, various apparatus may be installed. In FIG. 19, as an example, various apparatus group 5101 for endoscopic surgery, a ceiling camera 5187, a surgery field camera 5189, a plurality of display apparatus 5103A to 5103D, a recorder 5105, a patient bed 5183 and an illumination 5191 are depicted. The ceiling camera 5187 is provided on the ceiling of a surgery room and images the hands of a surgeon. The surgery field camera 5189 is provided on the ceiling of the surgery room and images a state of the entire surgery room.

Among the apparatus mentioned, the apparatus group 5101 belongs to an endoscopic surgery system 5113 hereinafter described and include an endoscope, a display apparatus which displays an image picked up by the endoscope and so forth. Various apparatus belonging to the endoscopic surgery system 5113 are referred to also as medical equipment. Meanwhile, the display apparatus 5103A to 5103D, the recorder 5105, the patient bed 5183 and the illumination 5191 are apparatus which are equipped, for example, in the surgery room separately from the endoscopic surgery system 5113. The apparatus which do not belong to the endoscopic surgery system 5113 are referred to also as non-medical equipment. The audiovisual controller 5107 and/or the surgery room controlling apparatus 5109 cooperatively control operation of the medical equipment and the non-medical equipment with each other.

The audiovisual controller 5107 integrally controls processes of the medical equipment and the non-medical equipment relating to image display. Specifically, each of the apparatus group 5101, the ceiling camera 5187 and the surgery field camera 5189 from among the apparatus provided in the surgery room system 5100 may be an apparatus having a function of sending information to be displayed during surgery (such information is hereinafter referred to as display information, and the apparatus mentioned is hereinafter referred to as apparatus of a sending source). Meanwhile, each of the display apparatus 5103A to 5103D may be an apparatus to which display information is outputted (the apparatus is hereinafter referred to also as apparatus of an output destination). Further, the recorder 5105 may be an apparatus which serves as both of an apparatus of a sending source and an apparatus of an output destination. The audiovisual controller 5107 has a function of controlling operation of an apparatus of a sending source and an apparatus of an output destination to acquire display information from the apparatus of a sending source and transmit the display information to the apparatus of an output destination so as to be displayed or recorded. It is to be noted that the display information includes various images picked up during surgery, various kinds of information relating to the surgery (for example, physical information of a patient, inspection results in the past or information regarding a surgical procedure) and so forth.

Specifically, to the audiovisual controller 5107, information relating to an image of a surgical region in a body lumen of a patient imaged by the endoscope may be transmitted as the display information from the apparatus group 5101. Further, from the ceiling camera 5187, information relating to an image of the hands of the surgeon picked up by the ceiling camera 5187 may be transmitted as display information. Further, from the surgery field camera 5189, information relating to an image picked up by the surgery field camera 5189 and illustrating a state of the entire surgery room may be transmitted as display information. It is to be noted that, if a different apparatus having an image pickup function exists in the surgery room system 5100, then the audiovisual controller 5107 may acquire information relating to an image picked up by the different apparatus as display information also from the different apparatus.

Alternatively, for example, in the recorder 5105, information relating to such images as mentioned above picked up in the past is recorded by the audiovisual controller 5107. The audiovisual controller 5107 can acquire, as display information, information relating to the images picked up in the past from the recorder 5105. It is to be noted that also various pieces of information relating to surgery may be recorded in advance in the recorder 5105.

The audiovisual controller 5107 controls at least one of the display apparatus 5103A to 5103D, which are apparatus of an output destination, to display acquired display information (namely, images picked up during surgery or various pieces of information relating to the surgery). In the example depicted, the display apparatus 5103A is a display apparatus installed so as to be suspended from the ceiling of the surgery room; the display apparatus 5103B is a display apparatus installed on a wall face of the surgery room; the display apparatus 5103C is a display apparatus installed on a desk in the surgery room; and the display apparatus 5103D is a mobile apparatus (for example, a tablet personal computer (PC)) having a display function.

Further, though not depicted in FIG. 19, the surgery room system 5100 may include an apparatus outside the surgery room. The apparatus outside the surgery room may be, for example, a server connected to a network constructed inside and outside the hospital, a PC used by medical staff, a projector installed in a meeting room of the hospital or the like. Where such an external apparatus is located outside the hospital, also it is possible for the audiovisual controller 5107 to cause display information to be displayed on a display apparatus of a different hospital through a teleconferencing system or the like to perform telemedicine.

The surgery room controlling apparatus 5109 integrally controls processes other than processes relating to image display on the non-medical equipment. For example, the surgery room controlling apparatus 5109 controls driving of the patient bed 5183, the ceiling camera 5187, the surgery field camera 5189 and the illumination 5191.

In the surgery room system 5100, a centralized operation panel 5111 is provided such that it is possible to issue an instruction regarding image display to the audiovisual controller 5107 or issue an instruction regarding operation of the non-medical equipment to the surgery room controlling apparatus 5109 through the centralized operation panel 5111. The centralized operation panel 5111 is configured by providing a touch panel on a display face of a display apparatus.

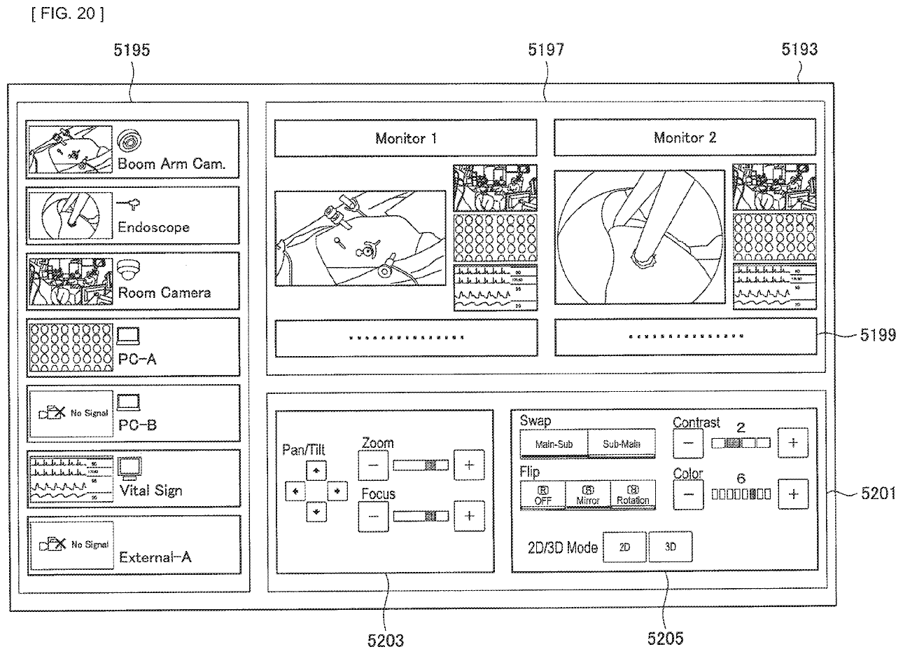

FIG. 20 is a view depicting an example of display of an operation screen image on the centralized operation panel 5111. In FIG. 20, as an example, an operation screen image is depicted which corresponds to a case in which two display apparatus are provided as apparatus of an output destination in the surgery room system 5100. Referring to FIG. 20, the operation screen image 5193 includes a sending source selection region 5195, a preview region 5197 and a control region 5201.

In the sending source selection region 5195, the sending source apparatus provided in the surgery room system 5100 and thumbnail screen images representative of display information the sending source apparatus have are displayed in an associated manner with each other. A user can select display information to be displayed on the display apparatus from any of the sending source apparatus displayed in the sending source selection region 5195.

In the preview region 5197, a preview of screen images displayed on two display apparatus (Monitor 1 and Monitor 2) which are apparatus of an output destination is displayed. In the example depicted, four images are displayed by picture in picture (PinP) display in regard to one display apparatus. The four images correspond to display information sent from the sending source apparatus selected in the sending source selection region 5195. One of the four images is displayed in a comparatively large size as a main image while the remaining three images are displayed in a comparatively small size as sub images. The user can exchange between the main image and the sub images by suitably selecting one of the images from among the four images displayed in the region. Further, a status displaying region 5199 is provided below the region in which the four images are displayed, and a status relating to surgery (for example, elapsed time of the surgery, physical information of the patient and so forth) may be displayed suitably in the status displaying region 5199.

A sending source operation region 5203 and an output destination operation region 5205 are provided in the control region 5201. In the sending source operation region 5203, a graphical user interface (GUI) part for performing an operation for an apparatus of a sending source is displayed. In the output destination operation region 5205, a GUI part for performing an operation for an apparatus of an output destination is displayed. In the example depicted, GUI parts for performing various operations for a camera (panning, tilting and zooming) in an apparatus of a sending source having an image pickup function are provided in the sending source operation region 5203. The user can control operation of the camera of an apparatus of a sending source by suitably selecting any of the GUI parts. It is to be noted that, though not depicted, where the apparatus of a sending source selected in the sending source selection region 5195 is a recorder (namely, where an image recorded in the recorder in the past is displayed in the preview region 5197), GUI parts for performing such operations as reproduction of the image, stopping of reproduction, rewinding, fast-feeding and so forth may be provided in the sending source operation region 5203.

Further, in the output destination operation region 5205, GUI parts for performing various operations for display on a display apparatus which is an apparatus of an output destination (swap, flip, color adjustment, contrast adjustment and switching between two dimensional (2D) display and three dimensional (3D) display) are provided. The user can operate the display of the display apparatus by suitably selecting any of the GUI parts.

It is to be noted that the operation screen image to be displayed on the centralized operation panel 5111 is not limited to the depicted example, and the user may be able to perform operation inputting to each apparatus which can be controlled by the audiovisual controller 5107 and the surgery room controlling apparatus 5109 provided in the surgery room system 5100 through the centralized operation panel 5111.

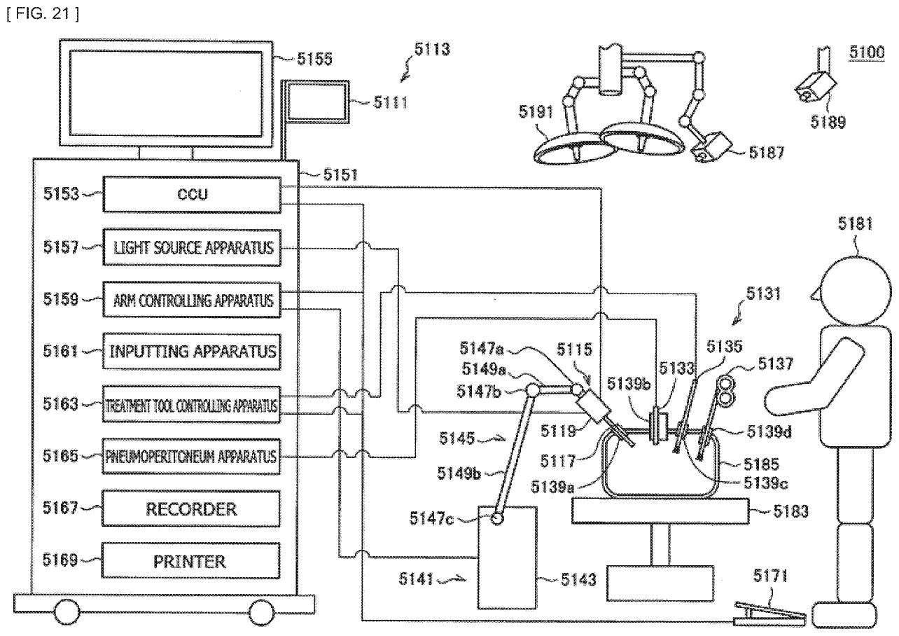

FIG. 21 is a view illustrating an example of a state of surgery to which the surgery room system described above is applied. The ceiling camera 5187 and the surgery field camera 5189 are provided on the ceiling of the surgery room such that it can image the hands of a surgeon (medical doctor) 5181 who performs treatment for an affected area of a patient 5185 on the patient bed 5183 and the entire surgery room. The ceiling camera 5187 and the surgery field camera 5189 may include a magnification adjustment function, a focal distance adjustment function, an imaging direction adjustment function and so forth. The illumination 5191 is provided on the ceiling of the surgery room and irradiates at least upon the hands of the surgeon 5181. The illumination 5191 may be configured such that the irradiation light amount, the wavelength (color) of the irradiation light, the irradiation direction of the light and so forth can be adjusted suitably.

The endoscopic surgery system 5113, the patient bed 5183, the ceiling camera 5187, the surgery field camera 5189 and the illumination 5191 are connected for cooperation with each other through the audiovisual controller 5107 and the surgery room controlling apparatus 5109 (not depicted in FIG. 21) as depicted in FIG. 19. The centralized operation panel 5111 is provided in the surgery room, and the user can suitably operate the apparatus existing in the surgery room through the centralized operation panel 5111 as described hereinabove.

In the following, a configuration of the endoscopic surgery system 5113 is described in detail. As depicted, the endoscopic surgery system 5113 includes an endoscope 5115, other surgical tools 5131, a supporting arm apparatus 5141 which supports the endoscope 5115 thereon, and a cart 5151 on which various apparatus for endoscopic surgery are mounted.

In endoscopic surgery, in place of incision of the abdominal wall to perform laparotomy, a plurality of tubular aperture devices called trocars 5139a to 5139d are used to puncture the abdominal wall. Then, a lens barrel 5117 of the endoscope 5115 and the other surgical tools 5131 are inserted into body lumens of the patient 5185 through the trocars 5139a to 5139d. In the example depicted, as the other surgical tools 5131, a pneumoperitoneum tube 5133, an energy treatment tool 5135 and forceps 5137 are inserted into body lumens of the patient 5185. Further, the energy treatment tool 5135 is a treatment tool for performing incision and peeling of a tissue, sealing of a blood vessel or the like by high frequency current or ultrasonic vibration. However, the surgical tools 5131 depicted are mere examples at all, and as the surgical tools 5131, various surgical tools which are generally used in endoscopic surgery such as, for example, a pair of tweezers or a retractor may be used.

An image of a surgical region in a body lumen of the patient 5185 picked up by the endoscope 5115 is displayed on a display apparatus 5155. The surgeon 5181 would use the energy treatment tool 5135 or the forceps 5137 while watching the image of the surgical region displayed on the display apparatus 5155 on the real time basis to perform such treatment as, for example, resection of an affected area. It is to be noted that, though not depicted, the pneumoperitoneum tube 5133, the energy treatment tool 5135, and the forceps 5137 are supported by the surgeon 5181, an assistant or the like during surgery.

### (Supporting Arm Apparatus)

The supporting arm apparatus 5141 includes an arm unit 5145 extending from a base unit 5143. In the example depicted, the arm unit 5145 includes joint portions 5147a, 5147b and 5147c and links 5149a and 5149b and is driven under the control of an arm controlling apparatus 5159. The endoscope 5115 is supported by the arm unit 5145 such that the position and the posture of the endoscope 5115 are controlled. Consequently, stable fixation in position of the endoscope 5115 can be implemented.

### (Endoscope)

The endoscope 5115 includes the lens barrel 5117 which has a region of a predetermined length from a distal end thereof to be inserted into a body lumen of the patient 5185, and a camera head 5119 connected to a proximal end of the lens barrel 5117. In the example depicted, the endoscope 5115 is depicted which is configured as a hard mirror having the lens barrel 5117 of the hard type. However, the endoscope 5115 may otherwise be configured as a soft mirror having the lens barrel 5117 of the soft type.

The lens barrel 5117 has, at a distal end thereof, an opening in which an objective lens is fitted. A light source apparatus 5157 is connected to the endoscope 5115 such that light generated by the light source apparatus 5157 is introduced to a distal end of the lens barrel 5117 by a light guide extending in the inside of the lens barrel 5117 and is applied toward an observation target in a body lumen of the patient 5185 through the objective lens. It is to be noted that the endoscope 5115 may be a direct view mirror or may be a perspective view mirror or a side view mirror.

An optical system and an image pickup element are provided in the inside of the camera head 5119 such that reflected light (observation light) from an observation target is condensed on the image pickup element by the optical system. The observation light is photo-electrically converted by the image pickup element to generate an electric signal corresponding to the observation light, namely, an image signal corresponding to an observation image. The image signal is transmitted as RAW data to a CCU 5153. It is to be noted that the camera head 5119 has a function incorporated therein for suitably driving the optical system of the camera head 5119 to adjust the magnification and the focal distance.

It is to be noted that, in order to establish compatibility with, for example, a stereoscopic vision (3D display), a plurality of image pickup elements may be provided on the camera head 5119. In this case, a plurality of relay optical systems are provided in the inside of the lens barrel 5117 in order to guide observation light to the plurality of respective image pickup elements.

### (Various Apparatus Incorporated in Cart)

The CCU 5153 includes a central processing unit (CPU), a graphics processing unit (GPU) or the like and integrally controls operation of the endoscope 5115 and the display apparatus 5155. Specifically, the CCU 5153 performs, for an image signal received from the camera head 5119, various image processes for displaying an image based on the image signal such as, for example, a development process (demosaic process). The CCU 5153 provides the image signal for which the image processes have been performed to the display apparatus 5155. Further, the audiovisual controller 5107 depicted in FIG. 19 is connected to the CCU 5153. The CCU 5153 provides the image signal for which the image processes have been performed also to the audiovisual controller 5107. Further, the CCU 5153 transmits a control signal to the camera head 5119 to control driving of the camera head 5119. The control signal may include information relating to an image pickup condition such as a magnification or a focal distance. The information relating to an image pickup condition may be inputted through the inputting apparatus 5161 or may be inputted through the centralized operation panel 5111 described hereinabove.

The display apparatus 5155 displays an image based on an image signal for which the image processes have been performed by the CCU 5153 under the control of the CCU 5153. If the endoscope 5115 is ready for imaging of a high resolution such as 4K (horizontal pixel number 3840× vertical pixel number 2160), 8K (horizontal pixel number 7680×vertical pixel number 4320) or the like and/or ready for 3D display, then a display apparatus by which corresponding display of the high resolution and/or 3D display are possible may be used as the display apparatus 5155. Where the apparatus is ready for imaging of a high resolution such as 4K or 8K, if the display apparatus used as the display apparatus 5155 has a size of equal to or not less than 55 inches, then a more immersive experience can be obtained. Further, a plurality of display apparatus 5155 having different resolutions and/or different sizes may be provided in accordance with purposes.

The light source apparatus 5157 includes a light source such as, for example, a light emitting diode (LED) and supplies irradiation light for imaging of a surgical region to the endoscope 5115.

The arm controlling apparatus 5159 includes a processor such as, for example, a CPU and operates in accordance with a predetermined program to control driving of the arm unit 5145 of the supporting arm apparatus 5141 in accordance with a predetermined controlling method.

An inputting apparatus 5161 is an input interface for the endoscopic surgery system 5113. A user can perform inputting of various kinds of information or instruction inputting to the endoscopic surgery system 5113 through the inputting apparatus 5161. For example, the user would input various kinds of information relating to surgery such as physical information of a patient, information regarding a surgical procedure of the surgery and so forth through the inputting apparatus 5161. Further, the user would input, for example, an instruction to drive the arm unit 5145, an instruction to change an image pickup condition (type of irradiation light, magnification, focal distance or the like) by the endoscope 5115, an instruction to drive the energy treatment tool 5135 or a like through the inputting apparatus 5161.

The type of the inputting apparatus 5161 is not limited and may be that of any one of various known inputting apparatus. As the inputting apparatus 5161, for example, a mouse, a keyboard, a touch panel, a switch, a foot switch 5171 and/or a lever or the like may be applied. Where a touch panel is used as the inputting apparatus 5161, it may be provided on the display face of the display apparatus 5155.

The inputting apparatus 5161 is otherwise a device to be mounted on a user such as, for example, a glasses type wearable device or a head mounted display (HMD), and various kinds of inputting are performed in response to a gesture or a line of sight of the user detected by any of the devices mentioned. Further, the inputting apparatus 5161 includes a camera which can detect a motion of a user, and various kinds of inputting are performed in response to a gesture or a line of sight of a user detected from a video picked up by the camera. Further, the inputting apparatus 5161 includes a microphone which can collect the voice of a user, and various kinds of inputting are performed by voice through the microphone. By configuring the inputting apparatus 5161 such that various kinds of information can be inputted in a contactless fashion in this manner, especially a user who belongs to a clean area (for example, the surgeon 5181) can operate an apparatus belonging to an unclean area in a contactless fashion. Further, since the user can operate an apparatus without releasing a possessed surgical tool from its hand, the convenience to the user is improved.

A treatment tool controlling apparatus 5163 controls driving of the energy treatment tool 5135 for cautery or incision of a tissue, sealing of a blood vessel or the like. A pneumoperitoneum apparatus 5165 feeds gas into a body lumen of the patient 5185 through the pneumoperitoneum tube 5133 to inflate the body lumen in order to secure the field of view of the endoscope 5115 and secure the working space for the surgeon. A recorder 5167 is an apparatus capable of recording various kinds of information relating to surgery. A printer 5169 is an apparatus capable of printing various kinds of information relating to surgery in various forms such as a text, an image or a graph.

In the following, especially a characteristic configuration of the endoscopic surgery system 5113 is described in more detail.

### (Supporting Arm Apparatus)

The supporting arm apparatus 5141 includes the base unit 5143 serving as a base, and the arm unit 5145 extending from the base unit 5143. In the example depicted, the arm unit 5145 includes the plurality of joint portions 5147a, 5147b and 5147c and the plurality of links 5149a and 5149b connected to each other by the joint portion 5147b. In FIG. 21, for simplified illustration, the configuration of the arm unit 5145 is depicted in a simplified form. Actually, the shape, number and arrangement of the joint portions 5147a to 5147c and the links 5149a and 5149b and the direction and so forth of axes of rotation of the joint portions 5147a to 5147c can be set suitably such that the arm unit 5145 has a desired degree of freedom. For example, the arm unit 5145 may preferably be included such that it has a degree of freedom equal to or not less than 6 degrees of freedom. This makes it possible to move the endoscope 5115 freely within the movable range of the arm unit 5145. Consequently, it becomes possible to insert the lens barrel 5117 of the endoscope 5115 from a desired direction into a body lumen of the patient 5185.

An actuator is provided in the joint portions 5147a to 5147c, and the joint portions 5147a to 5147c include such that they are rotatable around predetermined axes of rotation thereof by driving of the actuator. The driving of the actuator is controlled by the arm controlling apparatus 5159 to control the rotational angle of each of the joint portions 5147a to 5147c thereby to control driving of the arm unit 5145. Consequently, control of the position and the posture of the endoscope 5115 can be implemented. Thereupon, the arm controlling apparatus 5159 can control driving of the arm unit 5145 by various known controlling methods such as force control or position control.

For example, if the surgeon 5181 suitably performs operation inputting through the inputting apparatus 5161 (including the foot switch 5171), then driving of the arm unit 5145 may be controlled suitably by the arm controlling apparatus 5159 in response to the operation input to control the position and the posture of the endoscope 5115. After the endo scope 5115 at the distal end of the arm unit 5145 is moved from an arbitrary position to a different arbitrary position by the control just described, the endoscope 5115 can be supported fixedly at the position after the movement. It is to be noted that the arm unit 5145 may be operated in a master-slave fashion. In this case, the arm unit 5145 may be remotely controlled by the user through the inputting apparatus 5161 which is placed at a place remote from the surgery room.

Further, where force control is applied, the arm controlling apparatus 5159 may perform power-assisted control to drive the actuators of the joint portions 5147a to 5147c such that the arm unit 5145 may receive external force by the user and move smoothly following the external force. This makes it possible to move the arm unit 5145 with comparatively weak force when the user directly touches with and moves the arm unit 5145. Accordingly, it becomes possible for the user to move the endoscope 5115 more intuitively by a simpler and easier operation, and the convenience to the user can be improved.

Here, generally in endoscopic surgery, the endoscope 5115 is supported by a medical doctor called scopist. In contrast, where the supporting arm apparatus 5141 is used, the position of the endoscope 5115 can be fixed with a higher degree of certainty without hands, and therefore, an image of a surgical region can be obtained stably and surgery can be performed smoothly.

It is to be noted that the arm controlling apparatus 5159 may not necessarily be provided on the cart 5151. Further, the arm controlling apparatus 5159 may not necessarily be a single apparatus. For example, the arm controlling apparatus 5159 may be provided in each of the joint portions 5147a to 5147c of the arm unit 5145 of the supporting arm apparatus 5141 such that the plurality of arm controlling apparatus 5159 cooperate with each other to implement driving control of the arm unit 5145.

### (Light Source Apparatus)

The light source apparatus 5157 supplies irradiation light upon imaging of a surgical region to the endoscope 5115. The light source apparatus 5157 includes a white light source which includes, for example, an LED, a laser light source or a combination of them. In this case, where a white light source includes a combination of red, green, and blue (RGB) laser light sources, since the output intensity and the output timing can be controlled with a high degree of accuracy for each color (each wavelength), adjustment of the white balance of a picked up image can be performed by the light source apparatus 5157. Further, in this case, if laser beams from the RGB laser light sources are applied time-divisionally on an observation target and driving of the image pickup elements of the camera head 5119 is controlled in synchronism with the irradiation timings, then images individually corresponding to the R, G and B colors can be picked up time-divisionally. According to the method just described, a color image can be obtained even if a color filter is not provided for the image pickup element.

Further, driving of the light source apparatus 5157 may be controlled such that the intensity of light to be outputted is changed for each predetermined time. By controlling driving of the image pickup element of the camera head 5119 in synchronism with the timing of the change of the intensity of light to acquire images time-divisionally and synthesizing the images, an image of a high dynamic range free from underexposed blocked up shadows and overexposed highlights can be created.

Further, the light source apparatus 5157 may be configured to supply light of a predetermined wavelength band ready for special light observation. In special light observation, for example, by utilizing the wavelength dependency of absorption of light of a body tissue, narrow band light observation (narrow band imaging) of imaging a predetermined tissue such as a blood vessel of a superficial portion of the mucous membrane or the like in a high contrast is performed by applying light of a narrower band in comparison with irradiation light upon ordinary observation (namely, white light). Alternatively, in special light observation, fluorescent observation for obtaining an image from fluorescent light generated by irradiation of excitation light may also be performed. In fluorescent observation, it is possible to perform observation of fluorescent light from a body tissue by irradiating excitation light on the body tissue (autofluorescence observation) or to obtain a fluorescent light image by locally injecting a reagent such as indocyanine green (ICG) into a body tissue and irradiating excitation light corresponding to a fluorescent light wavelength of the reagent upon the body tissue. The light source apparatus 5157 can be configured to supply such narrow-band light and/or excitation light suitable for special light observation as described above.

### (Camera Head and CCU)

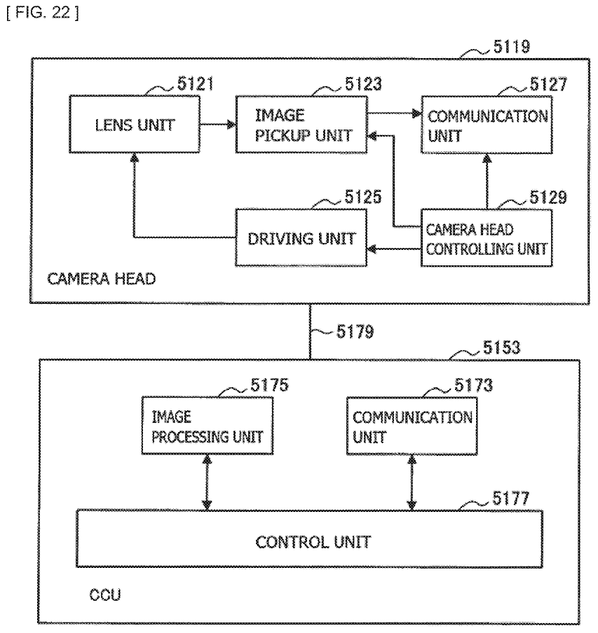

Functions of the camera head 5119 of the endoscope 5115 and the CCU 5153 are described in more detail with reference to FIG. 22. FIG. 22 is a block diagram depicting an example of a functional configuration of the camera head 5119 and the CCU 5153 depicted in FIG. 21.

Referring to FIG. 22, the camera head 5119 has, as functions thereof, a lens unit 5121, an image pickup unit 5123, a driving unit 5125, a communication unit 5127 and a camera head controlling unit 5129. Further, the CCU 5153 has, as functions thereof, a communication unit 5173, an image processing unit 5175 and a control unit 5177. The camera head 5119 and the CCU 5153 are connected to be bidirectionally communicable to each other by a transmission cable 5179.

First, a functional configuration of the camera head 5119 is described. The lens unit 5121 is an optical system provided at a connecting location of the camera head 5119 to the lens barrel 5117. Observation light taken in from a distal end of the lens barrel 5117 is introduced into the camera head 5119 and enters the lens unit 5121. The lens unit 5121 includes a combination of a plurality of lenses including a zoom lens and a focusing lens. The lens unit 5121 has optical properties adjusted such that the observation light is condensed on a light receiving face of the image pickup element of the image pickup unit 5123. Further, the zoom lens and the focusing lens include such that the positions thereof on their optical axis are movable for adjustment of the magnification and the focal point of a picked up image.

The image pickup unit 5123 includes an image pickup element and disposed at a succeeding stage to the lens unit 5121. Observation light having passed through the lens unit 5121 is condensed on the light receiving face of the image pickup element, and an image signal corresponding to the observation image is generated by photoelectric conversion. The image signal generated by the image pickup unit 5123 is provided to the communication unit 5127.

As the image pickup element which is included by the image pickup unit 5123, an image sensor, for example, of the complementary metal oxide semiconductor (CMOS) type is used which has a Bayer array and is capable of picking up an image in color. It is to be noted that, as the image pickup element, an image pickup element may be used which is ready, for example, for imaging of an image of a high resolution equal to or not less than 4K. If an image of a surgical region is obtained in a high resolution, then the surgeon 5181 can comprehend a state of the surgical region in enhanced details and can proceed with the surgery more smoothly.

Further, the image pickup element which is included by the image pickup unit 5123 is configured such that it has a pair of image pickup elements for acquiring image signals for the right eye and the left eye compatible with 3D display. Where 3D display is applied, the surgeon 5181 can comprehend the depth of a living body tissue in the surgical region with a higher degree of accuracy. It is to be noted that, if the image pickup unit 5123 is configured as that of the multi-plate type, then a plurality of systems of lens units 5121 are provided corresponding to the individual image pickup elements of the image pickup unit 5123.

The image pickup unit 5123 may not necessarily be provided on the camera head 5119. For example, the image pickup unit 5123 may be provided just behind the objective lens in the inside of the lens barrel 5117.

The driving unit 5125 includes an actuator and moves the zoom lens and the focusing lens of the lens unit 5121 by a predetermined distance along the optical axis under the control of the camera head controlling unit 5129. Consequently, the magnification and the focal point of a picked up image by the image pickup unit 5123 can be adjusted suitably.

The communication unit 5127 includes a communication apparatus for transmitting and receiving various kinds of information to and from the CCU 5153. The communication unit 5127 transmits an image signal acquired from the image pickup unit 5123 as RAW data to the CCU 5153 through the transmission cable 5179. Thereupon, in order to display a picked up image of a surgical region in low latency, preferably the image signal is transmitted by optical communication. This is because, since, upon surgery, the surgeon 5181 performs surgery while observing the state of an affected area through a picked up image, in order to achieve surgery with a higher degree of safety and certainty, it is demanded for a moving image of the surgical region to be displayed on the real time basis as far as possible. Where optical communication is applied, a photoelectric conversion module for converting an electric signal into an optical signal is provided in the communication unit 5127. After the image signal is converted into an optical signal by the photoelectric conversion module, it is transmitted to the CCU 5153 through the transmission cable 5179.

Further, the communication unit 5127 receives a control signal for controlling driving of the camera head 5119 from the CCU 5153. The control signal includes information relating to image pickup conditions such as, for example, information that a frame rate of a picked up image is designated, information that an exposure value upon image picking up is designated and/or information that a magnification and a focal point of a picked up image are designated. The communication unit 5127 provides the received control signal to the camera head controlling unit 5129. It is to be noted that also the control signal from the CCU 5153 may be transmitted by optical communication. In this case, a photoelectric conversion module for converting an optical signal into an electric signal is provided in the communication unit 5127. After the control signal is converted into an electric signal by the photoelectric conversion module, it is provided to the camera head controlling unit 5129.

It is to be noted that the image pickup conditions such as the frame rate, exposure value, magnification or focal point are set automatically by the control unit 5177 of the CCU 5153 on the basis of an acquired image signal. In other words, an auto exposure (AE) function, an auto focus (AF) function and an auto white balance (AWB) function are incorporated in the endoscope 5115.

The camera head controlling unit 5129 controls driving of the camera head 5119 on the basis of a control signal from the CCU 5153 received through the communication unit 5127. For example, the camera head controlling unit 5129 controls driving of the image pickup element of the image pickup unit 5123 on the basis of information that a frame rate of a picked up image is designated and/or information that an exposure value upon image picking up is designated. Further, for example, the camera head controlling unit 5129 controls the driving unit 5125 to suitably move the zoom lens and the focus lens of the lens unit 5121 on the basis of information that a magnification and a focal point of a picked up image are designated. The camera head controlling unit 5129 may include a function for storing information for identifying of the lens barrel 5117 and/or the camera head 5119.

It is to be noted that, by disposing the components such as the lens unit 5121 and the image pickup unit 5123 in a sealed structure having high airtightness and high waterproof, the camera head 5119 can be provided with resistance to an autoclave sterilization process.

Now, a functional configuration of the CCU 5153 is described. The communication unit 5173 includes a communication apparatus for transmitting and receiving various kinds of information to and from the camera head 5119. The communication unit 5173 receives an image signal transmitted thereto from the camera head 5119 through the transmission cable 5179. Thereupon, the image signal may be transmitted preferably by optical communication as described above. In this case, for the compatibility with optical communication, the communication unit 5173 includes a photoelectric conversion module for converting an optical signal into an electric signal. The communication unit 5173 provides the image signal after conversion into an electric signal to the image processing unit 5175.

Further, the communication unit 5173 transmits, to the camera head 5119, a control signal for controlling driving of the camera head 5119. Also the control signal may be transmitted by optical communication.

The image processing unit 5175 performs various image processes for an image signal in the form of RAW data transmitted thereto from the camera head 5119. The image processes include various known signal processes such as, for example, a development process, an image quality improving process (a bandwidth enhancement process, a super-resolution process, a noise reduction (NR) process and/or an image stabilization process) and/or an enlargement process (electronic zooming process). Further, the image processing unit 5175 performs a detection process for an image signal for performing AE, AF and AWB.

The image processing unit 5175 includes a processor such as a CPU or a GPU, and when the processor operates in accordance with a predetermined program, the image processes and the detection process described above can be performed. It is to be noted that, where the image processing unit 5175 includes a plurality of GPUs, the image processing unit 5175 suitably divides information relating to an image signal such that image processes are performed in parallel by the plurality of GPUs.

The control unit 5177 performs various kinds of control relating to image picking up of a surgical region by the endoscope 5115 and display of the picked up image. For example, the control unit 5177 generates a control signal for controlling driving of the camera head 5119. Thereupon, if image pickup conditions are inputted by the user, then the control unit 5177 generates a control signal on the basis of the input by the user. Alternatively, where the endoscope 5115 has an AE function, an AF function and an AWB function incorporated therein, the control unit 5177 suitably calculates an optimum exposure value, focal distance and white balance in response to a result of a detection process by the image processing unit 5175 and generates a control signal.

Further, the control unit 5177 controls the display apparatus 5155 to display an image of a surgical region on the basis of an image signal for which the image processes have been performed by the image processing unit 5175. Thereupon, the control unit 5177 recognizes various objects in the surgical region image using various image recognition technologies. For example, the control unit 5177 can recognize a surgical tool such as forceps, a particular living body region, bleeding, mist when the energy treatment tool 5135 is used and so forth by detecting the shape, color and so forth of edges of the objects included in the surgical region image. The control unit 5177 causes, when it controls the display apparatus 5155 to display a surgical region image, various kinds of surgery supporting information to be displayed in an overlapping manner with an image of the surgical region using a result of the recognition. Where surgery supporting information is displayed in an overlapping manner and presented to the surgeon 5181, the surgeon 5181 can proceed with the surgery more safety and certainty.

The transmission cable 5179 which connects the camera head 5119 and the CCU 5153 to each other is an electric signal cable ready for communication of an electric signal, an optical fiber ready for optical communication or a composite cable thereof.

Here, while, in the example depicted in the figure, communication is performed by wired communication using the transmission cable 5179, the communication between the camera head 5119 and the CCU 5153 may be performed otherwise by wireless communication. Where the communication between the camera head 5119 and the CCU 5153 is performed by wireless communication, there is no necessity to lay the transmission cable 5179 in the surgery room. Therefore, such a situation that movement of medical staff in the surgery room is disturbed by the transmission cable 5179 can be eliminated.

An example of the surgery room system 5100 to which the technology according to an embodiment of the present disclosure can be applied has been described above. It is to be noted here that, although a case in which the medical system to which the surgery room system 5100 is applied is the endoscopic surgery system 5113 has been described as an example, the configuration of the surgery room system 5100 is not limited to that of the example described above. For example, the surgery room system 5100 may be applied to a soft endoscopic system for inspection or a microscopic surgery system in place of the endoscopic surgery system 5113.

The technology according to an embodiment of the present disclosure can be suitably applied to, for example, the audiovisual controller 5107, among the above-described configurations. Specifically, the audiovisual controller 5107 may have the functions of the format acquisition section 112, the image analysis section 114, the output control section 116, and the like described above, and may cause output images to be outputted to reduce a difference among respective output images corresponding to a plurality of input images for comparison among the images.

In a case where the technology according to an embodiment of the present disclosure is applied to the audiovisual controller 5107, the input image may be an image acquired by imaging of a camera such as the ceiling camera 5187, the surgery field camera 5189, and the endoscope 5115, or an image stored in the recorder 5105. For example, the image acquired by the imaging of the surgery field cameras 5189 and the image acquired by the imaging of the endoscopes 5115 may be the input image. Alternatively, the image acquired by the imaging of the endoscope 5115 and an image acquired by imaging of an unillustrated microscope may be the input image. Alternatively, the image acquired by the imaging of the surgery field camera 5189 and an image acquired by imaging of an unillustrated line-of-sight camera (wearable camera) worn by the surgeon may be the input image.

Different types of cameras may be used in surgery in some instances. For example, types of images obtained by a microscope camera and an endoscope camera are different from each other. Therefore, in a case where different types of cameras are used for the same surgery target, comparing images obtained by imaging of different types of cameras for reproduction as described above brings an effect of being able to grasp the status of a surgery site more easily.

In addition, a wearable camera worn by the surgeon may be used in combination with the surgery field camera for recording, etc. of a surgery in some instances; however, as for an image acquired by imaging of the surgery field camera, there is a possibility that an appropriate image may not be able to be record in a case where the surgeon looks into a surgical region, in a case where the field of view is obstructed by other medical staff, or the like. Therefore, using the image of the wearable camera in combination makes it possible to compensate for a part which was not visible by the surgery field camera. Thus, by comparing the images acquired by each imaging of the surgery field camera 5189 and the wearable camera for reproduction as described above, it is possible, even in a case where one of the cameras was not able to acquire the image appropriately, to easily confirm an image of the other.

It is to be noted that the example of images to be compared is not limited to the above-described example. Various images that may be acquired or displayed during the surgery may be used as input images. In addition, the input images are not limited to two; three or more images (e.g., images acquired by imaging of three or more different cameras) may be used as the input images.

In addition, in a case where the technology according to an embodiment of the present disclosure is applied to the audiovisual controller 5107, the audiovisual controller 5107 may cause an output image to be outputted to reduce a difference in at least one of the format parameters, for example. In addition, the audiovisual controller 5107 may cause an output image to be outputted to reduce a difference in a subject parameter. It is to be noted that, here, the subject parameter may include, for example, a parameter for an angle of view indicating a range in which a subject is to be shot. In addition, the audiovisual controller 5107 may cause the output image to be outputted on the basis of a corresponding key frame among the plurality of input images. It is to be noted that, in such a case, for example, the moment of hemostasis may be used as a key frame, and slow reproduction and displaying may be performed before and after the key frame.

Applying the technology according to an embodiment of the present disclosure to the audiovisual controller 5107 makes it possible to make comparison more easily among images captured by a plurality of cameras during surgery, for example.

### 4. Configuration Example of Hardware

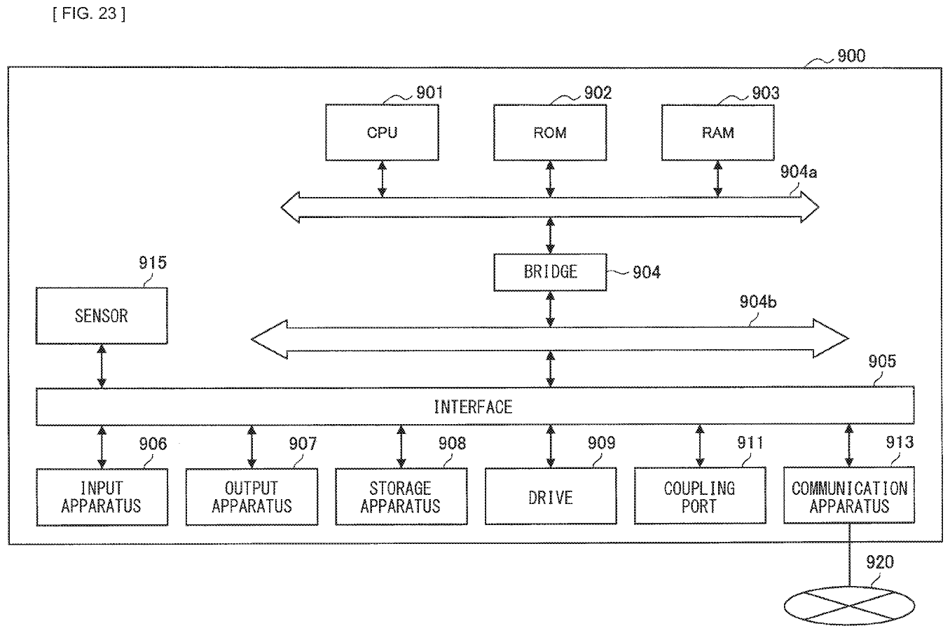

The description has been given above of the embodiments of the present disclosure. Finally, description is given of a hardware configuration of the information processor according to an embodiment of the present disclosure with reference to FIG. 23. FIG. 23 is a block diagram illustrating an example of the hardware configuration of the information processor according to an embodiment of the present disclosure. It is to be noted that an information processor 900 illustrated in FIG. 23 may achieve, for example, the information processor 1, the operation terminal 2, and the information processor 7 described above. Information processing by the information processor 1, the operation terminal 2, or the information processor 7 according to an embodiment of the present disclosure is achieved by cooperation between software and hardware described below.

As illustrated in FIG. 23, the information processor 900 includes a CPU (Central Processing Unit) 901, a ROM (Read Only Memory) 902, a RAM (Random Access Memory) 903, and a host bus 904a. In addition, the information processor 900 includes a bridge 904, an external bus 904b, an interface 905, an input apparatus 906, an output apparatus 907, a storage apparatus 908, a drive 909, a coupling port 911, a communication apparatus 913, and a sensor 915. The information processor 900 may include processing circuits such as a DSP or an ASIC in place of or in addition to the CPU 901.

The CPU 901 functions as an arithmetic processor and a controller, and controls overall operations in the information processor 900 in accordance with various programs. In addition, the CPU 901 may be a microprocessor. The ROM 902 stores programs to be used by the CPU 901, arithmetic parameters, and the like. The RAM 903 temporarily stores programs to be used in execution by the CPU 901, parameters appropriately changed in the execution, and the like. The CPU 901 may form, for example, the control unit 110, the control unit 210, or the control unit 710.

The CPU 901, the ROM 902 and the RAM 903 are coupled mutually by the host bus 904a including a CPU bus, or the like. The host bus 904a is coupled to the external bus 904b such as a PCI (Peripheral Component Interconnect/Interface) bus via the bridge 904. It is to be noted that it is not necessarily required to configure the host bus 904a, the bridge 904, and the external bus 904b to be separated; these functions may be implemented in one bus.

The input apparatus 906 may be achieved by, for example, an apparatus to which information is inputted by a user, such as a mouse, a keyboard, a touch panel, a button, a microphone, a switch, and a lever. In addition, the input apparatus 906 may be, for example, a remote control apparatus utilizing infrared rays or other radio waves, or may be an externally coupled apparatus such as a mobile phone or a PDA compatible with operations of the information processor 900. Further, the input apparatus 906 may include, for example, an input control circuit that generates an input signal on the basis of information inputted by a user who uses the input means described above and outputs the generated input signal to the CPU 901. By operating this input apparatus 906, the user of the information processor 900 is able to input various data to the information processor 900 or to give an instruction of a processing operation.

An output apparatus 907 is formed by an apparatus that is able to visually or auditorily notify the user of acquired information. Examples of such an apparatus include a display apparatus such as a CRT display apparatus, a liquid crystal display apparatus, a plasma display apparatus, an EL display apparatus, and a lamp, an audio output apparatus such as a speaker and a headphone, and a printing apparatus, etc. The output apparatus 907 outputs, for example, results obtained by various types of processing performed by the information processor 900. Specifically, the display apparatus visually displays the results obtained by various types of processing performed by the information processor 900 in various forms such as texts, images, tables, graphs, and the like. Meanwhile, the audio output apparatus converts an audio signal including reproduced audio data or acoustic data, etc. into an analog signal, and outputs the converted analog signal auditorily. The output apparatus 907 may form, for example, the display unit 240 or the display unit 740.

The storage apparatus 908 is an apparatus for storing data formed as an example of a storage unit of the information processor 900. The storage apparatus 908 is achieved by, for example, a magnetic storage unit device such as an HDD, a semiconductor storage device, an optical storage device, a magneto-optical storage device, or the like. The storage apparatus 908 may include a storage medium, a recording device that records data in the storage medium, a reading device that reads the data from the storage medium, a deleting device that deletes the data recorded in the storage medium, and the like. The storage apparatus 908 stores programs to be executed by the CPU 901, various data, various data acquired from the outside, and the like. The storage apparatus 908 may form, for example, the storage unit 150, the storage unit 250, or the storage unit 750.

The drive 909 is a reader/writer for a storage medium, and is built in or externally attached to the information processor 900. The drive 909 reads information recorded in an attached removable storage medium such as a magnetic disk, an optical disk, a magneto-optical disk, or a semiconductor memory, and outputs the read information to the RAM 903. In addition, the drive 909 is also able to write information into the removable storage medium.

The coupling port 911 is an interface to be coupled to an external apparatus, and is a coupling port with an external apparatus that is able to transmit data by, for example, a USB (Universal Serial Bus).

The communication apparatus 913 is, for example, a communication interface formed by a communication device for coupling to a network 920. The communication apparatus 913 is, for example, a communication card, etc. for wired or wireless LAN (Local Area Network), LTE (Long Term Evolution), Bluetooth (registered trademark), or WUSB (Wireless USB). In addition, the communication apparatus 913 may be a router for optical communication, a router for ADSL (Asymmetric Digital Subscriber Line), a modem for various types of communication, or the like. The communication apparatus 913 is able to transmit and receive signals or the like to and from the Internet or other communication apparatuses in accordance with a predetermined protocol such as TCP/IP, for example. The communication apparatus 913 may form, for example, the communication unit 120, the communication unit 220, or the communication unit 720.

The sensor 915 may be, for example, various sensors such as an acceleration sensor, a gyro sensor, a geomagnetic sensor, an optical sensor, a sound sensor, a ranging sensor, and a force sensor. The sensor 915 acquires information regarding a state of the information processor 900 itself, such as a posture and a moving speed of the information processor 900, and information regarding a surrounding environment of the information processor 900, such as brightness and noise around the information processor 900. In addition, the sensor 915 may include a GPS sensor that receives a GPS signal and measures the latitude, longitude, and altitude of the apparatus.

It is to be noted that the network 920 is a wired or wireless transmission path for information transmitted from an apparatus coupled to the network 920. For example, the network 920 may include a public network such as the Internet, a telephone network, a satellite communication network, and various types of LAN (Local Area Network) including Ethernet (registered trademark), WAN (Wide Area Network), and the like. In addition, the network 920 may include a private network such as IP-VPN (Internet Protocol-Virtual Private Network).

The description has been given above of an example of the hardware configuration that makes it possible to achieve the functions of the information processor 900 according to an embodiment of the present disclosure. Each of the above-described components may be achieved using general-purpose members, or may be achieved by hardware specialized in the functions of the respective components. Accordingly, it is possible to appropriately change hardware configurations to be utilized in accordance with a technical level at the time of implementing the embodiment of the present disclosure.

It is to be noted that it is possible to create a computer program for achieving each function of the information processor 900 according to an embodiment of the present disclosure as described above and to mount the computer program on a PC, etc. In addition, it is also possible to provide a computer-readable recording medium in which such a computer program is stored. The recording medium is, for example, a magnetic disk, an optical disk, a magneto-optical disk, a flash memory, or the like. In addition, the computer program described above may be distributed via a network, for example, without using a recording medium.

### 5. Closing

As described above, according to an embodiment of the present disclosure, it is possible to make comparison among a plurality of images more easily.

Although the description has been given above in detail of preferred embodiments of the present disclosure with reference to the accompanying drawings, the technical scope of the present disclosure is not limited to such examples. It is obvious that a person having ordinary skill in the art of the present disclosure may find various alterations or modifications within the scope of the technical idea described in the claims, and it should be understood that these alterations and modifications naturally come under the technical scope of the present disclosure.

In addition, the effects described herein are merely illustrative or exemplary, and are not limitative. That is, the technique according to the present disclosure may achieve, in addition to or in place of the above effects, other effects that are obvious to those skilled in the art from the description of the present specification.

It is to be noted that the technical scope of the present disclosure also includes the following configurations.

(1)

An information processor including an output control section that causes an output image obtained in a manner corresponding to each of a plurality of input images to be outputted to reduce a difference in at least one of subject parameters for a subject included in each of the plurality of input images.

(2)

The information processor according to (1), further including an image analysis section that acquires the subject parameters by analysis of the input images.

(3)

The information processor according to (1) or (2), in which the subject parameters include at least one of a dominant hand of the subject, a dominant foot of the subject, a size of the subject, a position of the subject in the input images, a distance from an imaging apparatus involved with imaging of the input images to the subject, or a posture of the subject with respect to the imaging apparatus.

(4)

The information processor according to any one of (1) to (3), in which the output control section causes the output image to be outputted on a basis of a key frame, the key frame being specified in each of the plurality of input images and corresponding among the plurality of input images.

(5)

The information processor according to (4), in which the output control section performs speed adjustment on the basis of the key frame to cause the output image to be outputted.

(6)

The information processor according to any one of (1) to (5), in which the output control section causes the output image to be outputted to reduce a difference in at least one of format parameters for an image format.

(7)

The information processor according to (6), in which the format parameters include at least one of a frame rate, resolution, or an aspect ratio.

(8)

The information processor according to any one of (1) to (7), in which the output control section causes the output image to be outputted on a basis of a condition set by a user.

(9)

The information processor according to (8), in which a parameter whose difference is reduced of the subject parameters is determined on the basis of the condition.

(10)

The information processor according to any one of (1) to (9), further including a storage unit that stores the subject parameters.

(11)

The information processor according to any one of (1) to (10), in which the output control section causes each output image to be outputted to separate apparatuses simultaneously.

(12)

The information processor according to any one of (1) to (11), in which the output control section causes each output image to be outputted to an identical apparatus simultaneously.

(13)

The information processor according to any one of (1) to (12), in which the output control section causes a plurality of the output images to be outputted in a superimposed manner.

(14)

The information processor according to any one of (1) to (13), further including:

a meta-information acquisition section that acquires meta-information regarding the input images; and

an image analysis section that specifies an extracted region on a basis of the meta-information acquired by the meta-information acquisition section and the input images, in which

the output control section causes the output image to be outputted on a basis of the extracted region.

(15)

The information processor according to (14), in which the meta-information includes event occurrence information regarding an event that has occurred in the input images, or subject information regarding the subject.

(16)

The information processor according to (14) or (15), in which the image analysis section acquires the meta-information not included in the meta-information acquired by the meta-information acquisition section by the analysis of the input images.

(17)

The information processor according to any one of (14) to (16), in which the image analysis section adjusts time information of the input images and time information of the meta-information acquired by the meta-information acquisition section by the analysis of the input images.

(18)

An information processing method including causing an output image obtained in a manner corresponding to each of a plurality of input images to be outputted to reduce a difference in at least one of subject parameters for a subject included in each of the plurality of input images.

(19)

A program that causes a computer to implement a function of causing an output image obtained in a manner corresponding to each of a plurality of input images to be outputted to reduce a difference in at least one of subject parameters for a subject included in each of the plurality of input images.

### REFERENCE NUMERALS LIST

1 Information processor
2 Operation terminal
3 Display apparatus
4 Imaging apparatus
5 Communication network
7 Information processor
110 Control unit
112 Format acquisition section
114 Image analysis section
116 Output control section
120 Communication unit
122 Format acquisition section
130 Display output interface unit
150 Storage unit
710 Control unit
712 Meta-information acquisition section
714 Image analysis section
716 Output control section
720 Communication unit
730 Operation unit
740 Display unit
750 Storage unit

1. An information processor comprising an output control section that causes an output image obtained in a manner corresponding to each of a plurality of input images to be outputted to reduce a difference in at least one of subject parameters for a subject included in each of the plurality of input images.

2. The information processor according to claim 1, further comprising an image analysis section that acquires the subject parameters by analysis of the input images.

3. The information processor according to claim 1, wherein the subject parameters include at least one of a dominant hand of the subject, a dominant foot of the subject, a size of the subject, a position of the subject in the input images, a distance from an imaging apparatus involved with imaging of the input images to the subject, or a posture of the subject with respect to the imaging apparatus.

4. The information processor according to claim 1, wherein the output control section causes the output image to be outputted on a basis of a key frame, the key frame being specified in each of the plurality of input images and corresponding among the plurality of input images.

5. The information processor according to claim 4, wherein the output control section performs speed adjustment on the basis of the key frame to cause the output image to be outputted.

6. The information processor according to claim 1, wherein the output control section causes the output image to be outputted to reduce a difference in at least one of format parameters for an image format.

7. The information processor according to claim 6, wherein the format parameters include at least one of a frame rate, resolution, or an aspect ratio.

8. The information processor according to claim 1, wherein the output control section causes the output image to be outputted on a basis of a condition set by a user.

9. The information processor according to claim 8, wherein a parameter whose difference is reduced of the subject parameters is determined on the basis of the condition.

10. The information processor according to claim 1, further comprising a storage unit that stores the subject parameters.

11. The information processor according to claim 1, wherein the output control section causes each output image to be outputted to separate apparatuses simultaneously.

12. The information processor according to claim 1, wherein the output control section causes each output image to be outputted to an identical apparatus simultaneously.

13. The information processor according to claim 1, wherein the output control section causes a plurality of the output images to be outputted in a superimposed manner.

14. The information processor according to claim 1, further comprising:
a meta-information acquisition section that acquires meta-information regarding the input images; and
an image analysis section that specifies an extracted region on a basis of the meta-information acquired by the meta-information acquisition section and the input images, wherein
the output control section causes the output image to be outputted on a basis of the extracted region.

15. The information processor according to claim 14, wherein the meta-information includes event occurrence information regarding an event that has occurred in the input images, or subject information regarding the subject.

16. The information processor according to claim 14, wherein the image analysis section acquires the meta-information not included in the meta-information acquired by the meta-information acquisition section by analysis of the input images.

17. The information processor according to claim 14, wherein the image analysis section adjusts time information of the input images and time information of the meta-information acquired by the meta-information acquisition section by analysis of the input images.

18. An information processing method comprising causing an output image obtained in a manner corresponding to each of a plurality of input images to be outputted to reduce a difference in at least one of subject parameters for a subject included in each of the plurality of input images.

19. A program that causes a computer to implement a function of causing an output image obtained in a manner corresponding to each of a plurality of input images to be outputted to reduce a difference in at least one of subject parameters for a subject included in each of the plurality of input images.
